(19) United States
(12) Patent Application Publication
(10) Pub. No.: US 2024/0180516 A1
(43) Pub. Date: Jun. 6, 2024

(54) WEARABLE SYSTEM FOR CARDIOVASCULAR MONITORING

(71) Applicant: InvenSense, Inc., San Jose, CA (US)

(72) Inventors: Leonardo BALDASARRE, Varese (IT); Marco TRAVAGLIATI, Pavia (IT); Enrico BONI, Firenze (IT); Giuseppe PASQUALINI, Monza (IT); Claudio SIMEONE, Brescia (IT)

(73) Assignee: InvenSense, Inc., San Jose, CA (US)

(21) Appl. No.: 18/527,223

(22) Filed: Dec. 1, 2023

Related U.S. Application Data
(60) Provisional application No. 63/385,956, filed on Dec. 2, 2022.
(60) Provisional application No. 63/479,983, filed on Jan. 13, 2023.
(60) Provisional application No. 63/490,769, filed on Mar. 16, 2023.

(51) Int. Cl. A61B 8/08; A61B 8/00

(52) U.S. Cl. CPC A61B 8/0891; A61B 8/0883; A61B 8/4227; A61B 8/4236; A61B 8/4494; A61B 8/5207; A61B 8/585; A61B 2562/227

(57) ABSTRACT

A wearable system for cardiovascular monitoring including an ultrasonic sensor module, a data storage unit, a processing unit, and a connection cable assembly for communicatively coupling the ultrasonic sensor module to the processing unit is described. The ultrasonic sensor module includes at least one array of ultrasonic transducers and at least one pre-amplification device coupled to the at least one array of ultrasonic transducers, the ultrasonic sensor module for placement on a human body proximate a blood vessel for performing cardiovascular monitoring. The processing unit includes hardware componentry for controlling transmission of ultrasonic signals and receipt of reflected ultrasonic signals at the at least one array of ultrasonic transducers, a digital processing module for performing on-board signal processing of the reflected ultrasonic signals received at the at least one array of ultrasonic transducers, and a power control system comprising an energy storage device for providing power to the system.

### RELATED APPLICATION

This application claims priority to and the benefit of co-pending U.S. Provisional Patent Application 63/385,956, filed on Dec. 2, 2022, entitled “WEARABLE PLATFORM FOR ULTRASOUND BIOSENSING,” by Baldasarre et al., having Attorney Docket No. IVS-1072-PR, and assigned to the assignee of the present application, which is incorporated herein by reference in its entirety.

This application also claims priority to and the benefit of co-pending U.S. Provisional Patent Application 63/479,983, filed on Jan. 13, 2023, entitled “SELF-ALIGNMENT OF TRANSVERSAL ULTRASOUND ARRAY,” by Colombo et al., having Attorney Docket No. IVS-1080-PR, and assigned to the assignee of the present application, which is incorporated herein by reference in its entirety.

This application also claims priority to and the benefit of co-pending U.S. Provisional Patent Application 63/490,769, filed on Mar. 16, 2023, entitled “MULTIPLE TRANSVERSAL ULTRASOUND ARRAYS FOR PULSE WAVE VELOCITY MEASUREMENTS,” by Baldasarre et al., having Attorney Docket No. IVS-1085-PR, and assigned to the assignee of the present application, which is incorporated herein by reference in its entirety.

### BACKGROUND

The development of consumer electronics has enabled the possibility to address the need of people's increasing awareness of their health and wellness. For example, wearable devices and smart phones have been able to host various sensor modalities for cardiovascular system monitoring, e.g., integrated electrodes for electrocardiogram (ECG), optical sensors for photoplethysmography (PPG), and pressure sensors for blood pressure. This enables people to measure parameters that can be used as an indicator for wellness themselves, for example at home without the need of a medical professional, or in the form of in-home care with the help of a medical professional. However, the ability of people to monitor their health and wellness, such as to monitor parameters of the cardiovascular system like electrical potential, pressure, or blood flow, depends on the available sensors, their ease of use, and their accuracy. Moreover, often the measurements are reflecting an averaged information over time or over (parts of) the body, lacking the details and/or fluctuations that may be useful for the monitoring process.

### BRIEF DESCRIPTION OF THE DRAWINGS

The accompanying drawings, which are incorporated in and form a part of the Description of Embodiments, illustrate various non-limiting and non-exhaustive embodiments of the subject matter and, together with the Description of Embodiments, serve to explain principles of the subject matter discussed below. Unless specifically noted, the drawings referred to in this Brief Description of Drawings should be understood as not being drawn to scale and like reference numerals refer to like parts throughout the various figures unless otherwise specified.

FIG. 1 illustrates a block diagram of an example system for cardiovascular monitoring, according to some embodiments.

FIGS. 2A and 2B illustrate block diagrams of example ultrasonic sensing modules of a system for cardiovascular monitoring, according to some embodiments.

FIGS. 3A, 3B, and 3C illustrate example arrays of ultrasonic transducers, according to some embodiments.

FIG. 3D illustrates an example array of ultrasonic transducers and signal lines of a connection cable, according to an embodiment.

FIG. 4 illustrates a block diagram of an example processing unit of a system for cardiovascular monitoring, according to some embodiments.

FIG. 5 illustrates a three-dimensional rendering of a system for cardiovascular monitoring comprising a processing unit with a System on Module (SoM) and componentry of an ultrasonic sensing module comprised within a flex-board and a rigid board, according to some embodiments.

FIGS. 6A through 6E illustrate graphical representations of the placement of ultrasonic sensing modules, connection cables, and processing unit on a human body, according to some embodiments.

FIGS. 7A and 7B illustrate different views of a graphical representation of an ultrasonic sensing system including two linear arrays of ultrasonic transducers for performing cardiovascular monitoring, according to some embodiments.

FIGS. 8A and 8B illustrate different views of a graphical representation of an ultrasonic sensing system including a two-dimensional array of ultrasonic transducers for performing cardiovascular monitoring, according to some embodiments.

FIGS. 9A, 9B, and 9C illustrate different views of graphical representations of automatic alignment of a transversal array of ultrasonic transducers, according to some embodiments.

FIGS. 10A and 10B illustrate different views of graphical representations of beamforming at a transversal array of ultrasonic transducers, according to some embodiments.

FIGS. 11A, 11B, 11C, and 11D illustrate different embodiments of performing automatic alignment of a transversal array, according to embodiments.

FIGS. 12A and 12B illustrates an example process for automatic alignment of a transversal array of ultrasonic transducers, according to some embodiments.

### DESCRIPTION OF EMBODIMENTS

The following Description of Embodiments is merely provided by way of example and not of limitation. Furthermore, there is no intention to be bound by any expressed or implied theory presented in the preceding background or in the following Description of Embodiments.

Reference will now be made in detail to various embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications and equivalents, which may be included within the spirit and scope the various embodiments as defined by the appended claims. Furthermore, in this Description of Embodiments, numerous specific details are set forth in order to provide a thorough understanding of embodiments of the present subject matter. However, embodiments may be practiced without these specific details. In other instances, well known methods, procedures, components, and circuits have not been described in detail as not to unnecessarily obscure aspects of the described embodiments.

### Notation and Nomenclature

Some portions of the detailed descriptions which follow are presented in terms of procedures, logic blocks, processing and other symbolic representations of operations on data within an electrical device. These descriptions and representations are the means used by those skilled in the data processing arts to most effectively convey the substance of their work to others skilled in the art. In the present application, a procedure, logic block, process, or the like, is conceived to be one or more self-consistent procedures or instructions leading to a desired result. The procedures are those requiring physical manipulations of physical quantities. Usually, although not necessarily, these quantities take the form of acoustic (e.g., ultrasonic) signals capable of being transmitted and received by an electronic device and/or electrical or magnetic signals capable of being stored, transferred, combined, compared, and otherwise manipulated in an electrical device.

It should be borne in mind, however, that all of these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “controlling,” “performing,” “determining,” “detecting,” “sensing,” “integrating,” “calculating,” “providing,” “receiving,” “analyzing,” “confirming,” “displaying,” “presenting,” “using,” “completing,” “instructing,” “comparing,” “correlating,” “executing,” “processing,” or the like, refer to the actions and processes of an electronic device such as an electrical device.

Embodiments described herein may be discussed in the general context of processor-executable instructions residing on some form of non-transitory processor-readable medium, such as program modules, executed by one or more computers or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, logic, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example ultrasonic sensing system and/or mobile electronic device described herein may include components other than those shown, including well-known components.

Various techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, perform one or more of the methods described herein. The non-transitory processor-readable data storage medium may form part of a computer program product, which may include packaging materials.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor.

Various embodiments described herein may be executed by one or more processors, such as one or more motion processing units (MPUs), sensor processing units (SPUs), host processor(s) or core(s) thereof, digital signal processors (DSPs), general purpose microprocessors, application specific integrated circuits (ASICs), application specific instruction set processors (ASIPs), field programmable gate arrays (FPGAs), a programmable logic controller (PLC), a complex programmable logic device (CPLD), a discrete gate or transistor logic, discrete hardware components, or any combination thereof designed to perform the functions described herein, or other equivalent integrated or discrete logic circuitry. The term “processor,” as used herein may refer to any of the foregoing structures or any other structure suitable for implementation of the techniques described herein. As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Moreover, processors can exploit nano-scale architectures such as, but not limited to, molecular and quantum-dot based transistors, switches and gates, in order to optimize space usage or enhance performance of user equipment. A processor may also be implemented as a combination of computing processing units.

In addition, in some aspects, the functionality described herein may be provided within dedicated software modules or hardware modules configured as described herein. Also, the techniques could be fully implemented in one or more circuits or logic elements. A general purpose processor may be a microprocessor, but in the alternative, the processor may be any conventional processor, controller, microcontroller, or state machine. A processor may also be implemented as a combination of computing devices, e.g., a combination of an SPU/MPU and a microprocessor, a plurality of microprocessors, one or more microprocessors in conjunction with an SPU core, MPU core, or any other such configuration.

### Overview of Discussion

Discussion begins with a description of an example wearable system for cardiovascular monitoring. An example system for automatic alignment of a transversal ultrasonic array, and example operations of such a system, are then described. An example system for transversal ultrasonic sensing for cardiovascular monitoring, and example operations of such a system, are then described.

Medical ultrasound technology is currently employed by medical professionals for imaging of the vascular system. Based on the images, medical professionals, such as ultrasound technicians, can deduce various forms of information regarding vascular health, such as vascular wall motion tracking, blood flow, or elastic properties of the soft tissues (e.g., elastography). Typical ultrasound systems currently in use are for clinical usage and meant to be operated by specially educated medical experts. Conventional medical ultrasonic systems typically include ultrasound probes with various shapes and form factors for different body parts, and often output images that are then analyzed further. In a clinical setting, the ultrasound system is used by a physician or clinician to align the probes to the physiological sites of interest and diagnose based on the static ultrasound imaging, Doppler imaging, and elastography. Due to the complexity of the biological system and usage of the ultrasound systems, extensive ultrasound imaging and medical training is needed for conventional ultrasound examination and diagnosis.

Technology development over the last decades has resulted in miniaturized ultrasonic transducers as well as ever-increasing data processing power and storage. An example of the currently available miniaturized ultrasonic transducers is the application of ultrasonic fingerprint sensors in mobile devices. Embodiments describe herein provide an ultrasonic sensor system for cardiovascular system monitoring. The described system provides a user friendly system that does not necessarily require operation by a trained medical professional, but due to system optimization and signal processing, allows for home usage outside of a medical establishment. For example, the described system can be used by people at home (without medical training), by in-home care personnel, or even by automated home robots or similar autonomous devices. The described system can measure and output various parameters of the blood vessels, e.g., blood vessel diameter and time variations, pulse wave velocity, blood pressure, etc. The described system may also output a wellness indicator based on these parameters, and this wellness indicator may be monitored over time.

Embodiments described herein provide a wearable system for cardiovascular monitoring including an ultrasonic sensor module, a data storage unit, a processing unit, and a connection cable assembly for communicatively coupling the ultrasonic sensor module to the processing unit. The processing unit includes hardware componentry for controlling transmission of ultrasonic signals at the at least one array of ultrasonic transducers and for controlling receipt of reflected ultrasonic signals at the at least one array of ultrasonic transducers, a digital processing module for performing on-board signal processing of the reflected ultrasonic signals received at the at least one array of ultrasonic transducers, and a power control system comprising an energy storage device for providing power to the hardware componentry, the digital processing module, and the ultrasonic sensor module. In some embodiments, the cardiovascular monitoring includes at least one of pulse wave velocity, pulse transit time, arterial diameter, arterial wall motion, arterial wall stiffness, heart rate, and blood pressure.

The ultrasonic sensor module includes at least one array of ultrasonic transducers and at least one pre-amplification device coupled to the at least one array of ultrasonic transducers, where the at least one array of ultrasonic transducers includes a plurality of ultrasonic transducers. The ultrasonic sensor module is for placement on a human body proximate a blood vessel for performing cardiovascular monitoring. In some embodiments, the ultrasonic sensor module includes a first array of ultrasonic transducers and a second array of ultrasonic transducers. In some embodiments, the first array of ultrasonic transducers and the second array of ultrasonic transducers are one-dimensional arrays of ultrasonic transducers. In some embodiments, the ultrasonic sensor module includes a flex-board sub-unit comprising the at least one array of ultrasonic transducers and a rigid board comprising the at least one pre-amplification device. In some embodiments, the ultrasonic sensor module further includes a light emitting diode for providing visual feedback for aligning the at least one array of ultrasonic transducers with a blood vessel.

In some embodiments, the ultrasonic sensor module further includes a motion sensor for performing motion sensing, wherein the hardware componentry is also for controlling operation of the motion sensor. In some embodiments, the digital processing module is also for correlating motion sensing data of the motion sensor with the reflected ultrasonic signals for use in the signal processing of the reflected ultrasonic signals. In some embodiments, the hardware componentry is for controlling transmission of the ultrasonic signals according to motion sensing data of the motion sensor.

In some embodiments, the ultrasonic sensor module further includes a force sensor, wherein the digital processing module is also for controlling operation of the force sensor, wherein the force sensor is for sensing an applied pressure between the ultrasonic sensor module and the human body for ensuring appropriate contact pressure between the ultrasonic sensor module and the human body. In some embodiments, the digital processing module also utilizes the applied pressure in performing the cardiovascular monitoring. It should be appreciated that other sensors can be used to sense an applied pressure between the ultrasonic sensor module and the human body, such as a pressure sensor, a strain gauge, etc.

In some embodiments, the wearable system includes a second ultrasonic sensor module for placement on the human body at a different location than the ultrasonic sensor module and proximate a blood vessel for performing cardiovascular monitoring, wherein the processing unit is configured to trigger the hardware componentry of the ultrasonic sensor module and the second ultrasonic sensor module for synchronization of the transmission of the ultrasonic signals and the receipt of the reflected ultrasonic between the ultrasonic sensor module and the second ultrasonic sensor module.

The processing unit includes hardware componentry for controlling transmission of ultrasonic signals at the at least one array of ultrasonic transducers and for controlling receipt of reflected ultrasonic signals at the at least one array of ultrasonic transducers, a digital processing module for performing on-board signal processing of the reflected ultrasonic signals received at the at least one array of ultrasonic transducers, and a power control system comprising an energy storage device for providing power to the hardware componentry, the digital processing module, and the ultrasonic sensor module. In some embodiments, the digital processing module further includes a wireless communication module for communicating output of the on-board signal processing to a remote computer system.

In some embodiments, the hardware componentry, the digital processing module, and the power control system of the processing unit are included within a system on a chip (SoC) comprising a processor and a field programmable gate array (FPGA) for performing the on-board signal processing. In some embodiments, the digital processing module is also for aligning the at least one array of ultrasonic transducers by selecting an ultrasonic transducer of the at least one array of ultrasonic transducers as an aligned ultrasonic transducer. In some embodiments, the processing unit further includes an activation timer configured for autonomous activation of the ultrasonic sensor module and the processing unit enabling the transmission of the ultrasonic signals and the receipt of the reflected ultrasonic signals.

The connection cable assembly is for enabling signal communication and power transmission between the ultrasonic sensor module and the processing unit. In some embodiments, the connection cable assembly includes a plurality of signal lines corresponding to each transmission signal and each received signal for each receive channel of the at least one array of ultrasonic transducers.

Other embodiments described herein provide an ultrasonic sensing system including a plurality of subsets of ultrasonic transducers for performing cardiovascular monitoring. The subsets of ultrasonic transducers are arranged transversally to the blood vessel(s) being sensed. In one embodiment, the subsets of ultrasonic transducers include one-dimensional arrays of ultrasonic transducers. In other embodiments, the subsets of ultrasonic transducers include two sub-apertures of a two-dimensional array of ultrasonic transducers. In some embodiments, the ultrasonic signals transmitted from the first subset of ultrasonic transducers and the second subset of ultrasonic transducers utilize a same frequency. The artery motion reconstructed from each subsets of ultrasonic transducers is then analyzed to extract the pulse transit time (PTT) from one subset of ultrasonic transducers to another and estimate of the pulse wave velocity (PWV) is derived as PWV=L/PTT, where L is the distance between the two transducers.

The ultrasonic sensing system includes a plurality of ultrasonic transducers for placement on a human body proximate a blood vessel, a hardware controller for controlling operation of the plurality of ultrasonic transducers, and a digital processing module for processing the reflected ultrasonic signals received at the first subset of ultrasonic transducers and the second subset of ultrasonic transducers for performing cardiovascular monitoring. In some embodiments, the cardiovascular monitoring comprises determination of a pulse transit time. In some embodiments, the cardiovascular monitoring comprises at least one of arterial diameter, arterial wall motion, arterial wall stiffness, heart rate, and blood pressure. In some embodiments, the blood vessel is a radial artery.

The plurality of ultrasonic transducers includes a first subset of ultrasonic transducers and a second subset of ultrasonic transducers, wherein the ultrasonic transducers of the first subset and the second subset are arranged linearly, wherein the first subset of ultrasonic transducers is positioned parallel to the second subset of ultrasonic transducers at a fixed separation distance, and wherein the first subset of ultrasonic transducers and second subset of ultrasonic transducers are positioned transversally to the blood vessel.

The hardware controller is configured to control transmission of ultrasonic signals from the first subset of ultrasonic transducers and the second subset of ultrasonic transducers and configured to control receipt of reflected ultrasonic signals at the first subset of ultrasonic transducers and the second subset of ultrasonic transducers, wherein the reflected ultrasonic signals sense movement of a wall of the blood vessel.

In some embodiments, the ultrasonic signals transmitted from the first subset of ultrasonic transducers and the second subset of ultrasonic transducers utilize a same frequency. In some embodiments, the first subset of ultrasonic transducers and the second subset of ultrasonic transducers are comprised within a two-dimensional array of ultrasonic transducers. In some embodiments, the first subset of ultrasonic transducers is comprised within a first one-dimensional array of ultrasonic transducers and the second subset of ultrasonic transducers is comprised within a second one-dimensional array of ultrasonic transducers. In some embodiments, the first one-dimensional array of ultrasonic transducers and the second one-dimensional array of ultrasonic transducers are comprised within the same circuit board.

In some embodiments, the reflected ultrasonic signals received at the first subset of ultrasonic transducers are received at the digital processing module using a first receive channel and the reflected ultrasonic signals received at the second subset of ultrasonic transducers are received at the digital processing module using a second receive channel. In some embodiments, the reflected ultrasonic signals are received at the digital processing module from the first receive channel and the second receive channel. In some embodiments, the digital signal processing module is configured to account for the phase shift between a plurality of instances of the reflected ultrasonic signals received from the first receive channel and to account for the phase shift between a plurality of instances of the reflected ultrasonic signals received from the second receive channel.

In some embodiments, the digital processing module is also for aligning the plurality of ultrasonic transducers by selecting an ultrasonic transducer of the first subset of ultrasonic transducers as a first aligned ultrasonic transducer and by selecting an ultrasonic transducer of the second subset of ultrasonic transducers as a second aligned ultrasonic transducer.

Embodiments described herein enable continuous cardiovascular monitoring using a wearable system for ultrasonic sensing of blood vessels. In order to provide such cardiovascular monitoring, the system described herein enables automatic alignment of ultrasonic sensors with the target blood vessel. The embodiments described herein provide for automatic alignment with underlying blood vessels by determining which ultrasonic transducer of an array of ultrasonic transducers placed transversely to the target blood vessel exhibits alignment with the target blood vessel. The described embodiments perform automatic alignment without the need for a human operator and/or without image analysis, allowing for a wearable system capable of cardiovascular monitoring to perform the described alignment automatically.

Other embodiments described herein provide a method of automatic alignment of a transversal array of ultrasonic transducers. A plurality of instances of an ultrasonic scanning operation using an array of ultrasonic transducers is performed, wherein each instance of the plurality of instances of the ultrasonic scanning operation includes a different sub-array of ultrasonic transducers of the array of ultrasonic transducers, where a sub-array includes a portion of ultrasonic transducers of the array of ultrasonic transducers, and wherein each instance of the ultrasonic scanning operation transmits an ultrasonic signal and receives at least one reflected ultrasonic signal. A signal amplitude and a time of flight are determined for each of the at least one reflected ultrasonic signal of each instance of the ultrasonic scanning operation. An ultrasonic transducer of the array of ultrasonic transducers is selected as exhibiting alignment with the target based at least in part on the signal amplitude and the time of flight for each of the at least one reflected ultrasonic signal of each instance of the ultrasonic scanning operation.

In some embodiments, the array of ultrasonic transducers is placed transversally relative to the target. In some embodiments, the sub-array of ultrasonic transducers includes one ultrasonic transducer, such that each instance of the ultrasonic scanning operation transmits the ultrasonic signal at the one ultrasonic transducer and receives the at least one reflected ultrasonic signal at the one ultrasonic transducer.

In some embodiments, the sub-array of ultrasonic transducers of each instance includes a plurality of ultrasonic transducers. In some embodiments, each instance of the ultrasonic scanning operation transmits the ultrasonic signal by beamforming using the plurality of ultrasonic transducers of the sub-array and receives the at least one reflected ultrasonic signal at one ultrasonic transducer of the plurality of ultrasonic transducers. In some embodiments, the beamforming is defined according to time delays and apodization parameters of the array of ultrasonic transducers. In some embodiments, each instance of the ultrasonic scanning operation transmits the ultrasonic signal by generating a plane-wave using the plurality of ultrasonic transducers of the sub-array and receives the at least one reflected ultrasonic signal at one ultrasonic transducer of the plurality of ultrasonic transducers. In some embodiments, the plane-wave generation is defined according to apodization parameters of the array of ultrasonic transducers.

In some embodiments, the ultrasonic transducer of the array of ultrasonic transducers selected as exhibiting alignment with the target exhibits the smallest time of flight of the times of flight of each instance of the plurality of instances of the ultrasonic scanning operation. In some embodiments, the ultrasonic transducer of the array of ultrasonic transducers selected as exhibiting alignment with the target exhibits the largest time of flight of the times of flight of each instance of the plurality of instances of the ultrasonic scanning operation. In some embodiments, the ultrasonic transducer of the array of ultrasonic transducers selected as exhibiting alignment with the target exhibits the largest signal amplitude of the signal amplitudes of each instance of the ultrasonic scanning operation. In some embodiments, the ultrasonic transducer of the array of ultrasonic transducers selected as exhibiting alignment with the target exhibits the smallest signal amplitude of the signal amplitudes of each instance of the ultrasonic scanning operation. In some embodiments, the ultrasonic transducer of the array of ultrasonic transducers selected as exhibiting alignment with the target exhibits the largest time of flight differential between a first reflected ultrasonic signal of the at least one reflected ultrasonic signal and a second reflected ultrasonic signal of the at least one reflected ultrasonic signal. In some embodiments, sequential pulses are transmitted for each instance of the plurality of instances of the ultrasonic scanning operation using each sub-array of ultrasonic transducers of the array of ultrasonic transducers, such that a reflected ultrasonic signal is received from the target for each transmitted pulse. In some embodiments, a time of flight differential between subsequent reflected ultrasonic signals for each sub-array is determined. The ultrasonic transducer of the array of ultrasonic transducers selected as exhibiting alignment with the target exhibits the largest time of flight differential between reflected ultrasonic signals received during two instances of the ultrasonic scanning operation for each sub-array. In some embodiments, the ultrasonic transducer of the array of ultrasonic transducers selected as exhibiting alignment with the target exhibits the largest velocity amplitude of a Doppler signal. In some embodiments, the ultrasonic transducer of the array of ultrasonic transducers selected as exhibiting alignment with the target exhibits the largest width of the velocity profile identified from a Doppler signal.

### Example Wearable System for Cardiovascular Monitoring

As the heart pumps the blood through the vascular system, a pressure wave runs along the blood vessels, which themselves are elastic and flexible. This pressure wave causes the elastic vessels to expand and contract as these pressure waves pass. As a result, there is an expansion wave running along the blood vessels with each heartbeat, where this pressure wave is referred to as the pulse. The pulse wave velocity (PWV) is the velocity at which the blood pressure wave propagates through the circulatory system.

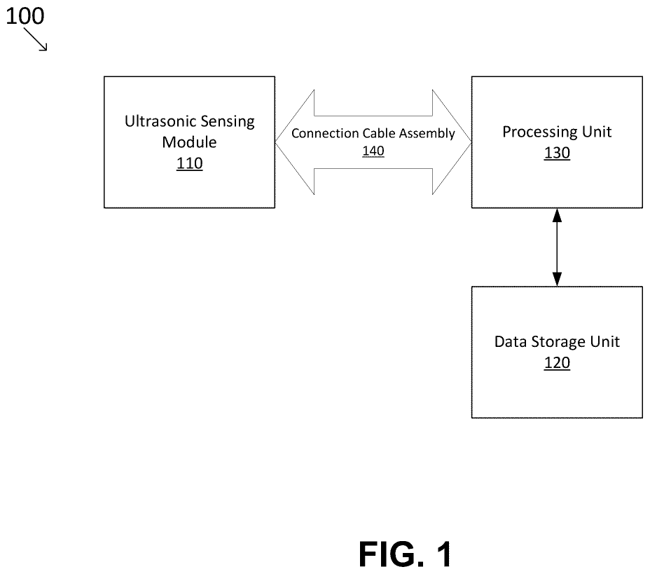

FIG. 1 illustrates a block diagram of an example ultrasonic sensing system 100 for cardiovascular monitoring, according to some embodiments. System 100 is configured to perform cardiovascular monitoring based at least in part on signals (e.g., acoustic signals) received from an ultrasonic sensor. Ultrasonic sensing system 100 includes ultrasonic sensing module 110, data storage unit 120, and processing unit 130, wherein ultrasonic sensing module 110 and processing unit 130 are communicatively coupled via connection cable assembly 140. Connection cable assembly 140 communicatively couples ultrasonic sensor module 110 to processing unit 130 and enables signal communication and power transmission between ultrasonic sensor module 110 and processing unit 130. While data storage unit 120 is illustrated as being external to processing unit 130, it should be appreciated that in some embodiments, data storage unit 120 may be integrated within processing unit 130. In other embodiments, data storage unit 120 may be distributed such that a portion of data storage unit 120 is integrated within processing unit 130 and a portion of data storage unit 120 is external to processing unit 130.

Ultrasonic sensing module 110 is for placement on a human body proximate a blood vessel for performing the cardiovascular monitoring. In some embodiments, the cardiovascular monitoring includes at least one of pulse wave velocity, pulse transit time, arterial diameter, arterial wall motion, arterial wall stiffness, heart rate, and blood pressure. Ultrasonic sensing module 110 includes at least one array of ultrasonic transducers and at least one pre-amplification device coupled to the at least one array of ultrasonic transducers, where the at least one array of ultrasonic transducers includes plurality of ultrasonic transducers. Processing unit 130 includes hardware componentry for controlling transmission of ultrasonic signals at ultrasonic sensing module 110 and for controlling receipt of reflected ultrasonic signals at ultrasonic sensing module 110, a digital processing module for performing on-board signal processing of the reflected ultrasonic signals, and a power control system including an energy storage device (e.g., a battery, a super capacitor, etc.) for providing power to the hardware componentry, the digital processing module, and ultrasonic sensor module 110.

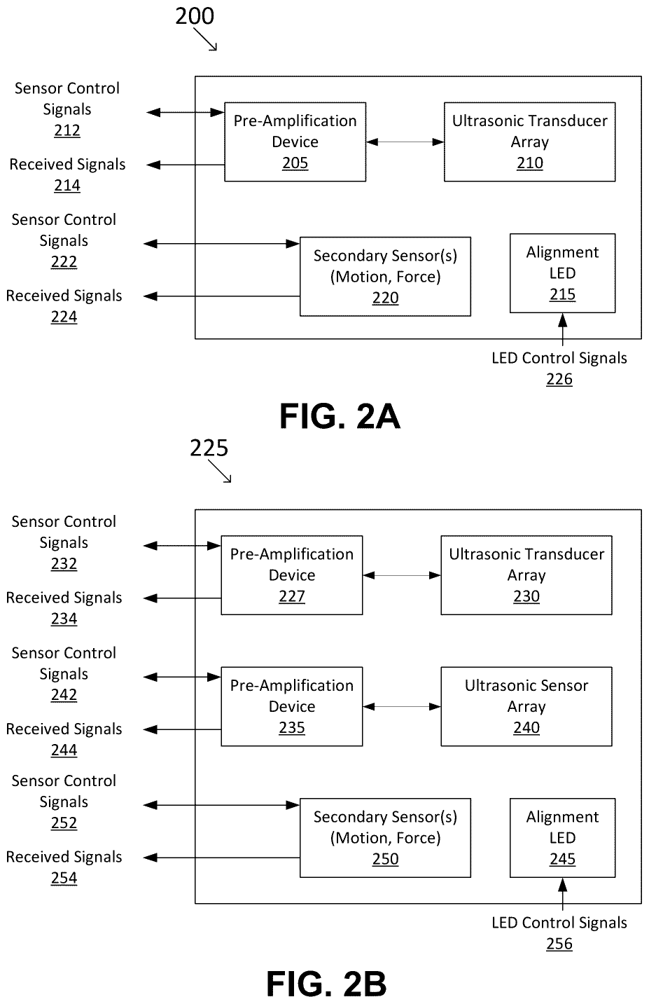

FIGS. 2A and 2B illustrate block diagrams of example ultrasonic sensing modules 200 and 225, respectively, of a system for cardiovascular monitoring (e.g., system 100), according to some embodiments. It should be appreciated that ultrasonic sensing module 110 of system 100 can be implemented as at least one of ultrasonic sensing modules 200 and 225. Ultrasonic sensing modules 200 and 225 are for placement on a human body proximate a blood vessel for performing cardiovascular monitoring.

With reference to FIG. 2A, ultrasonic sensing module 200 includes pre-amplification device 205 and ultrasonic transducer array 210. In some embodiments, ultrasonic sensing module 200 also include one or more secondary sensors 220, such as a motion sensor, a force sensor, or a temperature sensor. In some embodiments, ultrasonic sensing module 200 also includes alignment light emitting diode (LED) 215 for assisting in visual alignment of ultrasonic sensing module 200 with a blood vessel on a human body. LED control signals 226 are received at ultrasonic sensing module 200 for controlling operation of alignment LED 215. It should be appreciated that an optical feedback device other than an LED can be used (e.g., an optical sensor, a light, etc.) and that there can be one ore more optical feedback devices.

Sensor control signals 212 are received at ultrasonic sensing module 200 for controlling operation of ultrasonic transducer array 210. In some embodiments, pre-amplification device 205 receives sensor control signals 212 and converts sensor control signals 212 to have the appropriate signal strength for driving operation of ultrasonic transducers or ultrasonic transducer array 210. It should be appreciated that sensor control signals 212 include all signals for control operating of ultrasonic transducer array 210, including drive signals, power control, etc. In some embodiments, pre-amplification device 205 is configured to buffer weak received signals 214 received at ultrasonic transducer array 210. Sensor control signals 212 also include a transmit/receive switch control signal for switching operation of the ultrasonic transducer array 210 between a signal transmit mode and a signal receive mode.

Ultrasonic transducer array 210 is operable to emit and detect ultrasonic waves (also referred to as ultrasonic signals or ultrasound signals). One or more ultrasonic transducers (e.g., Piezoelectric Micromachined Ultrasonic Transducers (PMUTs)), which may be comprised within ultrasonic transducer array 210 may be used to transmit and receive the ultrasonic waves, where the ultrasonic transducers are capable of performing both the transmission and receipt of the ultrasonic waves. The emitted ultrasonic waves are reflected from any objects in contact with (or in front of) ultrasonic transducer array 210, and can project into the object at various depths, and these reflected ultrasonic waves, or echoes, are then detected and received at ultrasonic transducer array 210 as received signals 214. Where the object is a human body (e.g., at an arm or a wrist), the waves are projected into the tissue of the human body, and reflect at different tissue depths due to acoustic impedance mismatches.

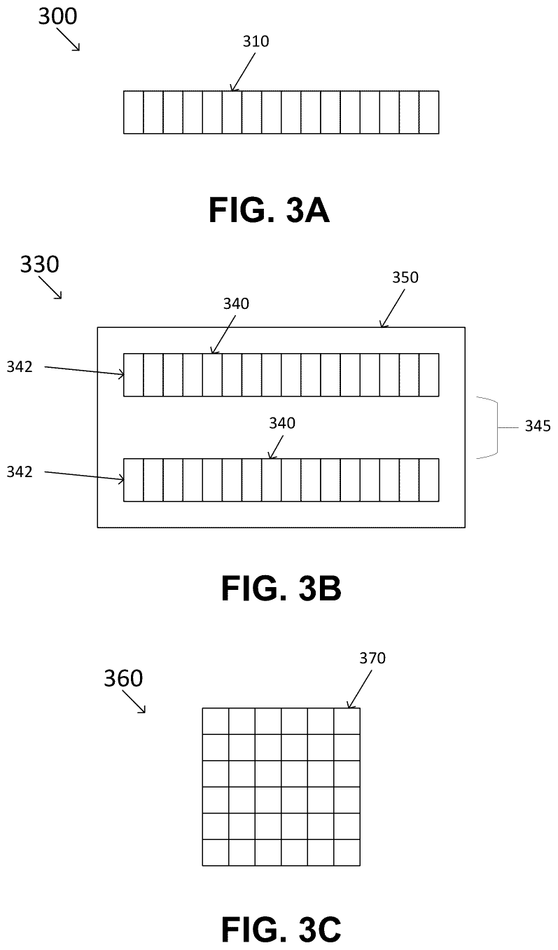

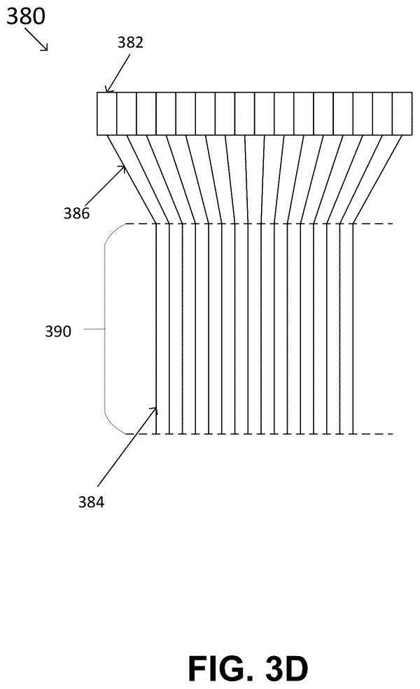

In some embodiments, ultrasonic transducer array 210 is a one-dimensional array of ultrasonic transducers. In some embodiments, ultrasonic transducer array 210 is a two-dimensional array of ultrasonic transducers. FIGS. 3A, 3B, and 3C illustrate examples of ultrasonic transducer arrays 300, 330, and 360, respectively, according to some embodiments. It should be appreciated that ultrasonic transducer array 210 can be implemented as at least one of ultrasonic transducer arrays 300, 330, and 360.

FIG. 3A illustrates ultrasonic transducer array 300 including a one-dimensional linear array of ultrasonic transducers 310. It should be appreciated that ultrasonic transducers 310 can be of any shape or size, e.g., square, rectangular, circular, etc.

FIG. 3B illustrates ultrasonic transducer array 330 including two one-dimensional linear arrays 342 of ultrasonic transducers 340, where one-dimensional linear arrays 342 are parallel and separate by a known separation distance 345. In some embodiments, one-dimensional linear arrays 342 are coupled to a board or substrate 350. It should be appreciated that ultrasonic transducers 340 can be of any shape or size, e.g., square, rectangular, circular, etc.

FIG. 3C illustrates ultrasonic transducer array 360 including a two-dimensional array of ultrasonic transducers 370. It should be appreciated that ultrasonic transducers 370 can be of any shape or size, e.g., square, rectangular, circular, etc. In accordance with various embodiments, rows or columns of ultrasonic transducers 370 of two-dimensional ultrasonic transducer array 360 can be operated as separate one-dimensional linear arrays of ultrasonic transducers. Moreover, in some embodiments, at least one inactive row/column of ultrasonic transducers 370 can separate two active rows/columns, thereby separating two active rows/columns by a known separation distance that is the width of the ultrasonic transducers 370 separating the active rows/columns. In other embodiments, neighboring rows/columns are used as separate one-dimensional linear arrays of ultrasonic transducers, where the pitch of the two-dimensional ultrasonic transducer array 360 is the known separation distance.

In some embodiments, each ultrasonic transducer of ultrasonic transducer array 210 has a dedicated signal line for communicating control signals to the ultrasonic transducer and communicating received ultrasonic signals for signal processing. FIG. 3D illustrates an example ultrasonic transducer array 380 and signal lines of a connection cable 390, according to an embodiment. Ultrasonic transducer array 380 is a linear array of ultrasonic transducers 382, where each ultrasonic transducer 382 is coupled to a dedicated signal line 384. Connection cable 390 (e.g., connection cable assembly 140 of FIG. 1) includes all signal lines 384. In some embodiments, a signal line 384 is coupled to its corresponding ultrasonic transducer 382 via electrical connection 386.

With reference to FIG. 2A, ultrasonic sensing module 200 is aligned with the ultrasound transducers in contact with the body on the spot proximate a blood vessel for which cardiovascular monitoring is to be performed. It should be appreciated that ultrasonic sensing module 200 is a system that might be physically divided into interconnected sub-units. For example, ultrasonic sensing module 200 can be comprised of a flex-board and a rigid board: the flex-board including ultrasonic transducer array 210 and the rigid-board including pre-amplification device 205. The rigid board may also include voltage regulators to derive all the voltages required to run the pre-amplification device 205 and bias the transducers of ultrasonic transducer array 210.

Ultrasonic transducer array 210 may be used for forming and steering an ultrasonic beam. The beam forming can be used to focus the ultrasonic waves at the correct depth, and the beam steering may be used to control lateral motion of the beam to find the blood vessel. For example, when the sensor is placed on the skin, the sensor may not be exactly above the blood vessel. The beam steering and beamforming may be used to find the vessel in a first step through a scanning action, and once the vessel is located, in a second step perform the blood vessel and blood flow measurements. The beam forming and beam steering can be accomplished by applying small phase or time delays to the individual transducers. The ultrasonic transducers may be controlled individually, or the ultrasonic transducers may be grouped together in subsets of transducers. These subset of ultrasonic transducers may be independently and/or collectively controlled. This may be done for generating the ultrasonic beam (transmit beamforming), but it may also help with the signal analysis of the detected reflected waves (receive beamforming). Location of the blood vessel may also be based on Doppler measurements or by looking for signal with the right heartbeat signal or frequency components. Furthermore, optimizing for a maximum change in amplitude can be used to determine the center middle of the blood vessel.

In some embodiments, ultrasonic sensing module 200 includes at least one secondary sensor 220, such as a motion sensor, a force sensor, or a temperature sensor. Sensor control signals 222 are received, e.g., from processing unit 130 of system 100, at secondary sensor 220 for controlling the operation of secondary sensor 220. It should be appreciated that sensor control signals 222 include all signals for controlling operation of secondary sensor 220, including drive signals, power control, etc. Responsive to performing sensing operations, received signals 224 are transmitted from secondary sensor 220 for processing and analysis, e.g., to processing unit 130.

In some embodiments, ultrasonic sensing module 110 includes two ultrasonic transducer arrays. With reference to FIG. 2B, ultrasonic sensing module 225 includes pre-amplification device 227 coupled to ultrasonic transducer array 230 and pre-amplification device 235 coupled to ultrasonic transducer array 240. It should be appreciated that, in some embodiments, pre-amplification device 227 and pre-amplification device 235 are combined into a single pre-amplification device coupled to both ultrasonic transducer array 230 and ultrasonic transducer array 240. In some embodiments, ultrasonic sensing module 225 also include one or more secondary sensors 250, such as a motion sensor, a force sensor, or a temperature sensor. In some embodiments, ultrasonic sensing module 225 also includes alignment LED 245 for assisting in visual alignment of ultrasonic sensing module 225 with a blood vessel on a human body. LED control signals 256 are received at ultrasonic sensing module 200 for controlling operation of alignment LED 245

Sensor control signals 232 are received at ultrasonic sensing module 225 for controlling operation of ultrasonic transducer array 230. In some embodiments, pre-amplification device 227 receives sensor control signals 232 and converts sensor control signals 232 to have the appropriate signal strength for driving operation of ultrasonic transducers or ultrasonic transducer array 230. It should be appreciated that sensor control signals 232 include all signals for control operating of ultrasonic transducer array 230, including drive signals, power control, etc. In some embodiments, pre-amplification device 227 is configured to buffer weak received signals 234 received at ultrasonic transducer array 230. Sensor control signals 232 also include a transmit/receive switch control signal for switching operation of the ultrasonic transducer array 230 between a signal transmit mode and a signal receive mode. Sensor control signals 242 are received at ultrasonic sensing module 225 for controlling operation of ultrasonic transducer array 230. In some embodiments, pre-amplification device 235 receives sensor control signals 242 and converts sensor control signals 242 to have the appropriate signal strength for driving operation of ultrasonic transducers or ultrasonic transducer array 240. It should be appreciated that sensor control signals 242 include all signals for control operating of ultrasonic transducer array 240, including drive signals, power control, etc. In some embodiments, pre-amplification device 235 is configured to buffer weak received signals 244 received at ultrasonic transducer array 240. Sensor control signals 242 also include a transmit/receive switch control signal for switching operation of the ultrasonic transducer array 240 between a signal transmit mode and a signal receive mode.

Ultrasonic transducer arrays 230 and 240 operable to emit and detect ultrasonic waves (also referred to as ultrasonic signals or ultrasound signals). One or more ultrasonic transducers (e.g., PMUTs), which may be comprised within ultrasonic transducer array 210 may be used to transmit and receive the ultrasonic waves, where the ultrasonic transducers are capable of performing both the transmission and receipt of the ultrasonic waves. The emitted ultrasonic waves are reflected from any objects in contact with (or in front of) ultrasonic transducer arrays 230 and 240, and can project into the object at various depths, and these reflected ultrasonic waves, or echoes, are then detected and received at ultrasonic transducer array 230 as received signals 234 and at ultrasonic transducer array 240 as received signals 244. Where the object is a human body (e.g., at an arm or a wrist), the waves are projected into the tissue of the human body, and reflect at different tissue depths due to acoustic impedance mismatches.

It should be appreciated that ultrasonic transducer array 230 and ultrasonic transducer array 240 can be one-dimensional array of ultrasonic transducers or two-dimensional array of ultrasonic transducers. FIGS. 3A, 3B, and 3C illustrate examples of ultrasonic transducer arrays 300, 330, and 360, respectively, according to some embodiments. It should be appreciated that ultrasonic transducer array 230 and ultrasonic transducer array 240 can be implemented as at least one of ultrasonic transducer arrays 300, 330, and 360. In some embodiments, each ultrasonic transducer of ultrasonic transducer array 230 and ultrasonic transducer array 240 has a dedicated signal line for communicating control signals to the ultrasonic transducer and communicating received ultrasonic signals for signal processing (e.g., as illustrated in FIG. 3D).

Ultrasonic sensing module 225 is aligned with the ultrasound transducers in contact with the body on the spot proximate a blood vessel for which cardiovascular monitoring is to be performed. It should be appreciated that ultrasonic sensing module 225 is a system that might be physically divided into interconnected sub-units. For example, ultrasonic sensing module 225 can be comprised of a flex-board and a rigid board: the flex-board including ultrasonic transducer array 230 and ultrasonic transducer array 240 and the rigid-board including pre-amplification device 227 and pre-amplification device 235. The rigid board may also include voltage regulators to derive all the voltages required to run the pre-amplification device 227 and pre-amplification device 235 and bias the transducers of ultrasonic transducer array 230 and ultrasonic transducer array 240.

Ultrasonic transducer array 230 and ultrasonic transducer array 240 may be used for forming and steering an ultrasonic beam. The beam forming can be used to focus the ultrasonic waves at the correct depth, and the beam steering may be used to control lateral motion of the beam to find the blood vessel. For example, when the sensor is placed on the skin, the sensor may not be exactly above the blood vessel. The beam steering and beamforming may be used to find the vessel in a first step through a scanning action, and once the vessel is located, in a second step perform the blood vessel and blood flow measurements. The beam forming and beam steering can be accomplished by applying small phase delays to the individual transducers. The ultrasonic transducers may be controlled individually, or the ultrasonic transducers may be grouped together in subsets of transducers. These subset of ultrasonic transducers may be independently and/or collectively controlled. This may be done for generating the ultrasonic beam (transmit beamforming), but it may also help with the signal analysis of the detected reflected waves (receive beamforming). Location of the blood vessel may also be based on Doppler measurements or by looking for signal with the right heartbeat signal or frequency components. Furthermore, optimizing for a maximum change in amplitude can be used to determine the center middle of the blood vessel.

In some embodiments, ultrasonic sensing module 225 includes at least one secondary sensor 250, such as a motion sensor, a force sensor, or a temperature sensor. Sensor control signals 252 are received, e.g., from processing unit 130 of system 100, at secondary sensor 250 for controlling the operation of secondary sensor 250. It should be appreciated that sensor control signals 252 include all signals for control operating of secondary sensor 250, including drive signals, power control, etc. Responsive to performing sensing operations, received signals 254 are transmitted from secondary sensor 250 for processing and analysis, e.g., to processing unit 130.

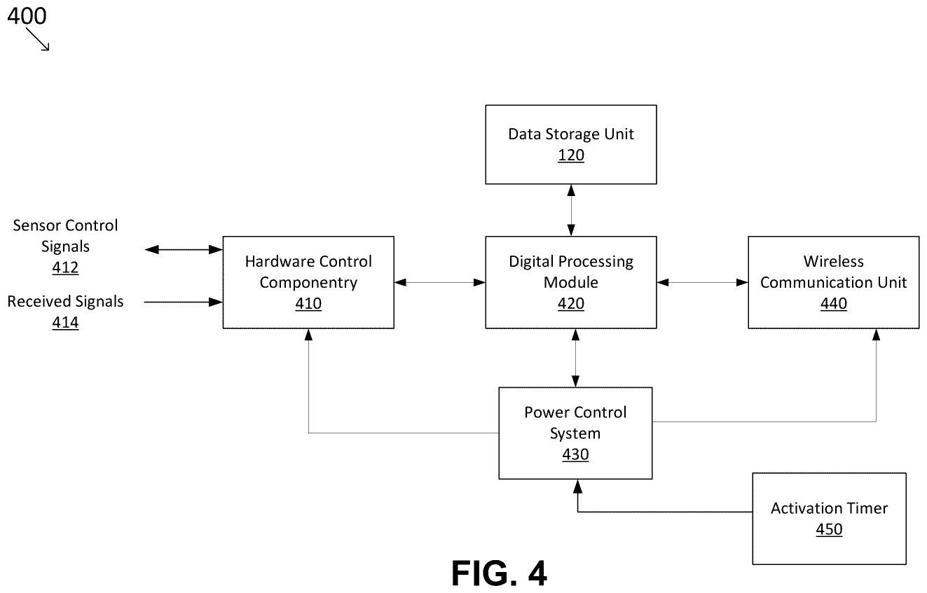

FIG. 4 illustrates a block diagram of an example processing unit 400 of a system for cardiovascular monitoring (e.g., system 100), according to some embodiments. In accordance with some embodiments, processing unit 400 is implemented as processing unit 130 of system 100. Processing unit 400 includes hardware control componentry 410, digital processing module 420, and power control system 430. In some embodiments, processing unit 400 includes wireless communication unit 440. It should also be appreciated that hardware control componentry 410, digital processing module 420, and power control system 430 may be separate components, may be comprised within a single component, or may be comprised in various combinations of multiple components, in accordance with some embodiments.

Data storage unit 120 is communicatively coupled to digital processing module 420. It should be appreciated that in accordance with some embodiments, data storage unit 120 may be integrated within processing unit 400, may be external to processing unit 400, or may be distributed such that a portion of data storage unit 120 is integrated within processing unit 400 and a portion of data storage unit 120 is external to processing unit 400.

Hardware control componentry 410 is configured to control operation of at least one ultrasonic sensor (e.g., ultrasonic sensing module 110, ultrasonic sensing module 200, or ultrasonic sensing module 225). For instance, hardware control componentry 410 is configured to communicate sensor control signals 412 to an ultrasonic sensor to control transmission of ultrasonic signals at an array of ultrasonic transducers of the ultrasonic sensor and to control receipt of reflected ultrasonic signals at the array of ultrasonic transducers. It should be appreciated that hardware control componentry 410 can perform other control operations, such as signal amplification, analog to digital conversion, and other functionality. Received signals 414 are received at hardware control componentry 410 for communication to and analysis by digital processing module 420. Hardware control componentry 410 includes voltage transmitters to drive the ultrasonic sensing module, controlling signal transmission strength, frequency, and activation as sensor control signals 412. In some embodiments, sensor control signals are transmitted via a cable connection (e.g., connection cable assembly 140).

Hardware control componentry 410 is configured to activate ultrasonic transducers of at least one ultrasonic sensor (e.g., an array of ultrasonic transducers) to perform ultrasonic signal transmission and receipt of reflected ultrasonic signals. In some embodiments, hardware control componentry 410 transmits sensor control signals 412 for individually activating and operating ultrasonic transducers of an ultrasonic transducer array. In other embodiments, hardware control componentry 410 transmits sensor control signals 412 for collectively operating a subset of ultrasonic transducers of an ultrasonic transducer array to perform beamforming and/or beam steering of an ultrasonic beam. For instance, sensor control signals 412 may delay activation of some ultrasonic transducers of an ultrasonic transducer array relative to other ultrasonic transducers, to focus a transmit beam to a particular location on or within the human body.

In some embodiments, hardware control componentry 410 is configured to intelligently manage the operation of the ultrasonic sensing module by controlling activation and signal receipt according to motion sensing data of the motion sensor. For example, motion sensing data may be received as received signals 414. Where the motion data indicates that the ultrasonic sensing module is in motion (e.g., received signals 224 from secondary sensor 220), and thus might not have stable placement on the human body proximate a blood vessel, hardware control componentry 410 can cease or refrain from commencing ultrasonic signal transmission and acquisition, thereby avoiding acquisition of erroneous or imprecise data.

In some embodiments, hardware control componentry 410 is configured to manage the operation of the ultrasonic sensing module by controlling activation and signal receipt according to force sensing data of the force sensor. For example, force sensing data may be received as received signals 414. Where the force data indicates that an applied pressure between the ultrasonic sensor module is below a threshold value indicative of appropriate contact pressure between the ultrasonic sensing module and the human body proximate a blood vessel, hardware control componentry 410 can cease or refrain from commencing ultrasonic signal transmission and acquisition, thereby avoiding acquisition of erroneous or imprecise data. It should be appreciated that other sensors can be used to sense an applied pressure between the ultrasonic sensor module and the human body, such as a pressure sensor, a strain gauge, etc.

Digital processing module 420 is configured to perform on-board signal processing of the received signals 414 received at the ultrasonic sensing module. In some embodiments, the data processing is performed on digital processing module 420 and can be a simple signal processing operation, or a complex pipeline with multiple steps of signal processing, e.g., utilizing artificial intelligence (AI) and machine learning algorithms. Digital processing module 420 also allows for the programming of different modalities of continuous monitoring, allowing for the control of parameters such as measurement frequency, ultrasonic schemes, etc. In some embodiments, processing unit 400 includes activation timer 450 configured providing for autonomous activation of the componentry of processing unit 400 enabling the transmission of the ultrasonic signals and the receipt of the reflected ultrasonic, power management operations, and digital processing operations signals. It should be appreciated that activation timer 450 may include componentry such as a microcontroller that activates the componentry of processing unit 400 periodically (e.g., every 10 minutes, every 30 minutes, every hour, etc.) For instance, subsequent processing unit 400 obtaining measurements for cardiovascular monitoring, processing unit 400 may turn off with the exception of activation timer 450, which consumes little power. Periodically, activation timer 450 can activate the componentry of processing unit 400 and an ultrasonic sensing module (e.g., ultrasonic sensing module 110) for performing further cardiovascular monitoring.

In some embodiments, digital processing module 420 is configured to perform signal processing in consideration of motion sensing data received from the motion sensor. For example, motion sensing data may be received as received signals 414. Digital processing module 420 is configured to correlate the motion sensing data with received ultrasonic signals, thereby identifying whether and how motion of the ultrasonic sensing module has impacted the received ultrasonic signals during acquisition.

In some embodiments, digital processing module 420 is configured to perform signal processing in consideration of force sensing data (e.g., applied pressure) received from the force sensor. For example, force sensing data may be received as received signals 414. Digital processing module 420 is configured to correlate the force sensing data with received ultrasonic signals, thereby identifying whether and how contact between the ultrasonic sensing module and the human body might have impacted the received ultrasonic signals during acquisition.

Power control system 430 is configured to provide power to hardware control componentry 410, digital processing module 420, and the ultrasonic sensing module (e.g., via connection cable assembly 140). In some embodiments, power control system 430 includes an energy storage device (e.g., a battery, a super capacitor, etc.) as a power supply. In some embodiments, digital processing module 400 further includes wireless communication unit 440 for communicating output of the on-board signal processing to a remote computer system for further analysis and processing.

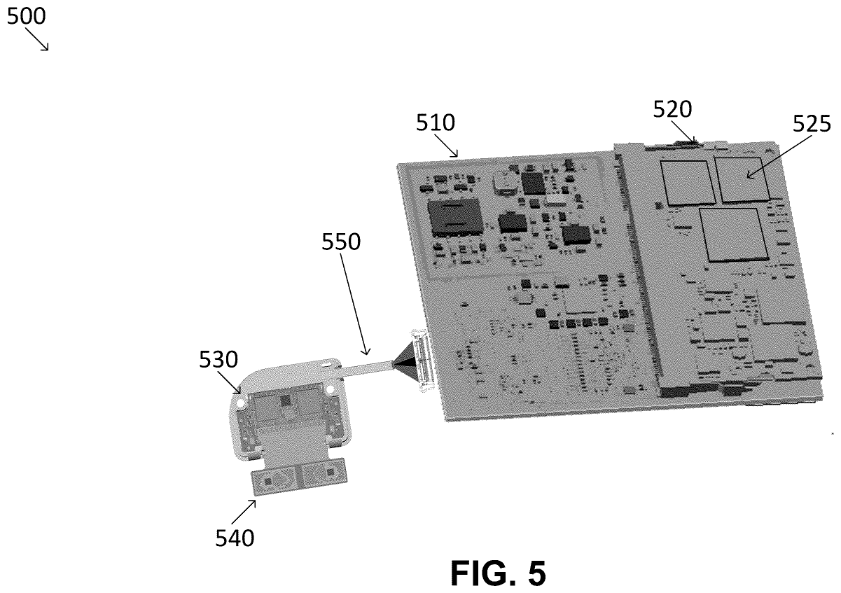

FIG. 5 illustrates a three dimensional rendering 500 of componentry of the described system for cardiovascular monitoring, according to some embodiments. FIG. 5 illustrates a three dimensional rendering of a system for cardiovascular monitoring comprising a processing unit with a System on Module (SoM) and componentry of an ultrasonic sensing module comprised within a flex-board and a rigid board, according to some embodiments As illustrated in diagram 500, hardware componentry 510 and digital processing module within System on Module (SoM) 520 including a System on a Chip (SoC) 525, where the SoC 525 includes the ultrasonic signal transmission and receipt clocking, receive signal processing, power management, an energy storage device, clock generation, and a field programmable gate array (FPGA) for performing the on-board signal processing. It should be appreciated that SoC 525, the FPGA, or any other processor or microcontroller can operate independently or collectively in various combinations to perform operations associated with cardiovascular monitoring. The ultrasonic sensor module includes flex-board sub-unit 540 comprising at least one array of ultrasonic transducers as well as any other sensors that might be included (e.g., a motion sensor, force sensor, etc.) and a rigid board 530 comprising at least one pre-amplification device. Connection cable assembly 550 communicatively couples the ultrasonic sensor module to the processing unit, where connection cable assembly 550 is for enabling signal communication and power transmission between the ultrasonic sensor module and the processing unit. It should be appreciated that the components are not necessarily shown to scale, and that the length of connection cable assembly 550 can vary according to application and location of the ultrasonic sensor module on the body relative to the processing unit.

The system for cardiovascular monitoring described herein is capable of being worn on a human body as a wearable system that allows for continuous capture of ultrasound measurements for prolonged times. For example, the wearable system for cardiovascular monitoring described herein can be worn in the same fashion of cardiac Holter monitors, ambulatory blood pressure monitoring systems or continuous pulse oximeters, also enabling spot-check measurements as many of these other monitoring systems do. The described embodiments can require little user interaction during use, and are capable of continuous monitoring, periodic monitoring, and also enabling beat-to-beat information retrieval during cardiovascular monitoring.

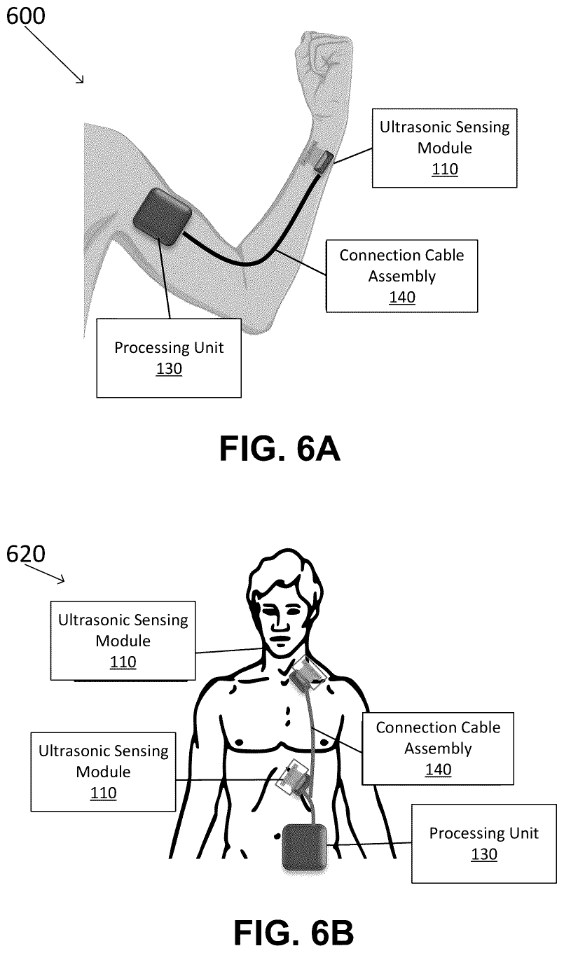

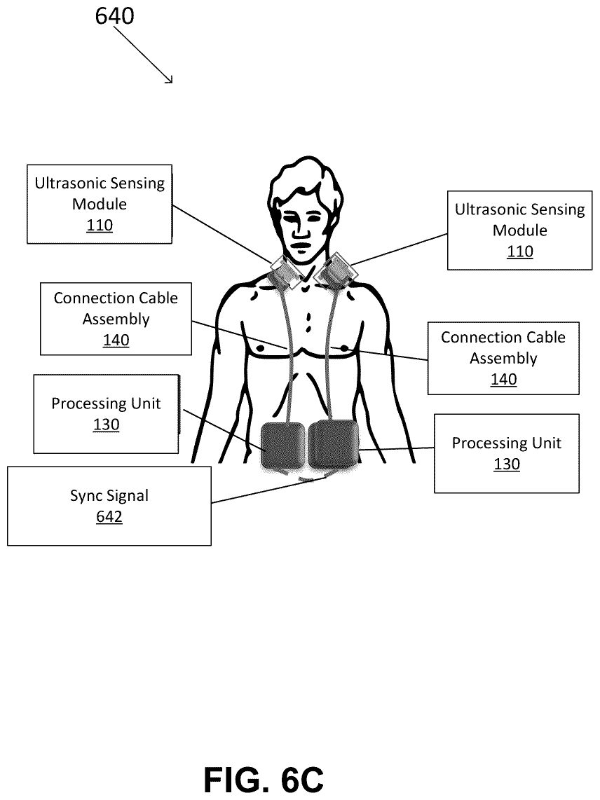

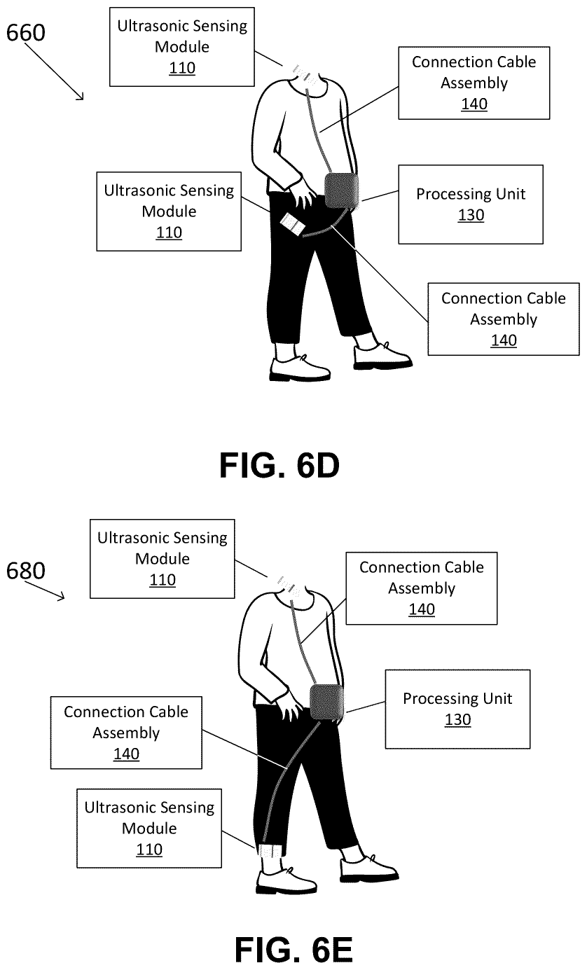

FIGS. 6A through 6E illustrate graphical representations of the placement of at least one ultrasonic sensing module 110, at least one connection cable assembly 140, and at least one processing unit 130 on a human body, according to some embodiments. It should be appreciated that ultrasonic sensing module 110 can be held in place using an adhesive patch, a strap, an elastic band, or any other attachment means for maintaining contact between the skin and ultrasonic sensing module 110. In some embodiments, ultrasonic sensing module 110 may also have a contact surface to improve conduction of the ultrasound waves into the skin of the user. The contact surface may comprise a gel like compartment, or other material, to increase the acoustic coupling. It should be further appreciated that other placements and use cases of the components of a wearable system for cardiovascular monitoring are possible, of which the illustrated embodiments are examples.

FIG. 6A illustrates placement 600 of the components of a wearable system for cardiovascular monitoring. As illustrated, in placement 600, ultrasonic sensing module 110 is placed on the wrist of a user, proximate the radial artery. In placement 600, processing unit 130 is worn on the arm of the user, and held in place using an adhesive patch, a strap, an elastic band, or any other attachment means. Connection cable assembly 140 runs along the user's arm and is coupled to ultrasonic sensing module 110 and processing unit 130. It should be appreciated that connection cable assembly 140 may be held in place by an attachment means, such as an adhesive, a strap, an elastic band, etc.

FIG. 6B illustrates placement 620 of the components of a wearable system for cardiovascular monitoring, where the wearable system includes two ultrasonic sensing modules 110. As illustrated, in placement 620, one ultrasonic sensing module 110 is placed on the neck of a user, proximate the common carotid artery, and another ultrasonic sensing module is placed on the abdomen of the user, proximate the abdominal aorta. In placement 600, processing unit 130 is worn on the lower abdomen of the user, and held in place using an adhesive patch, a strap, an elastic band, or any other attachment means. One connection cable assembly 140 runs along the abdomen and chest and is coupled to ultrasonic sensing module 110 near the carotid artery and processing unit 130. The other connection cable assembly 140 runs along the abdomen and is coupled to ultrasonic sensing module 110 near the abdominal aorta and processing unit 130. It should be appreciated that connection cable assemblies 140 may be held in place by an attachment means, such as an adhesive, a strap, an elastic band, etc.

In the embodiment of FIG. 6B, the wearable system for cardiovascular monitoring includes two ultrasonic sensing modules 110 under the control and operation of one processing unit 130. Where there are at least two ultrasonic sensing modules 110 under the control and operation of one processing unit 130, processing unit 130 is configured to trigger the hardware componentry of the ultrasonic sensor modules for synchronization of the transmission of the ultrasonic signals and the receipt of the reflected ultrasonic signals between the first ultrasonic sensor module and the second ultrasonic sensor module.

FIG. 6C illustrates placement 640 of the components of two wearable systems for cardiovascular monitoring synchronous operation. As illustrated, in placement 640, the ultrasonic sensing modules 110 are placed on placed on opposite side of the neck of a user, proximate each common carotid artery. In placement 640, both processing units 130 are worn on either side of the lower abdomen of the user, and held in place using an adhesive patch, a strap, an elastic band, or any other attachment means. Both connection cable assemblies 140 run along the abdomen and chest and are coupled to ultrasonic sensing modules 110 near the carotid artery and a processing unit 130. It should be appreciated that connection cable assemblies 140 may be held in place by an attachment means, such as an adhesive, a strap, an elastic band, etc. In placement 640, the processing units 130 are communicatively coupled via sync signal 642, wherein synchronization (sync) signal 642 coordinates synchronous clocking between the processing unites 130.

FIG. 6D illustrates placement 660 of the components of a wearable system for cardiovascular monitoring, where the wearable system includes two ultrasonic sensing modules 110. As illustrated, in placement 660, one ultrasonic sensing module 110 is placed on the neck of a user, proximate the common carotid artery, and another ultrasonic sensing module is placed near the hip of the user, proximate the femoral artery. In placement 660, processing unit 130 is worn on the lower abdomen of the user, and held in place using an adhesive patch, a strap, an elastic band, or any other attachment means. One connection cable assembly 140 runs along the abdomen and chest and is coupled to ultrasonic sensing module 110 near the carotid artery and processing unit 130. The other connection cable assembly 140 runs along the abdomen and hip and is coupled to ultrasonic sensing module 110 near the femoral artery and processing unit 130. It should be appreciated that connection cable assemblies 140 may be held in place by an attachment means, such as an adhesive, a strap, an elastic band, etc.

FIG. 6E illustrates placement 680 of the components of a wearable system for cardiovascular monitoring, where the wearable system includes two ultrasonic sensing modules 110. As illustrated, in placement 680, one ultrasonic sensing module 110 is placed on the neck of a user, proximate the common carotid artery, and another ultrasonic sensing module is placed near the ankle of the user, proximate to the ankle posterior tibial artery or dorsalis pedis artery. In placement 680, processing unit 130 is worn on the lower abdomen of the user, and held in place using an adhesive patch, a strap, an elastic band, or any other attachment means. One connection cable assembly 140 runs along the abdomen and chest and is coupled to ultrasonic sensing module 110 near the carotid artery and processing unit 130. The other connection cable assembly 140 runs along the abdomen and leg and is coupled to ultrasonic sensing module 110 near the ankle posterior tibial artery or dorsalis pedis artery and processing unit 130. It should be appreciated that connection cable assemblies 140 may be held in place by an attachment means, such as an adhesive, a strap, an elastic band, etc.

### Example Pulse Transit Time and Pulse Wave Velocity Measurements Using Transversal Arrays of Ultrasonic Transducers

Embodiments described herein provide a system for cardiovascular monitoring comprising at least two subsets of ultrasonic transducers positioned to record the pulsatile motion on different sites of the arterial tree (or the same artery). The system includes a plurality of subsets of ultrasonic transducers for performing cardiovascular monitoring. The ultrasonic sensing system includes a plurality of ultrasonic transducers for placement on a human body proximate a blood vessel, a hardware controller for controlling operation of the plurality of ultrasonic transducers, and a digital processing module for processing the reflected ultrasonic signals received at the first subset of ultrasonic transducers and the second subset of ultrasonic transducers for performing cardiovascular monitoring. The subsets of ultrasonic transducers are arranged transversally to the blood vessel(s) being sensed. In one embodiment, the subsets of ultrasonic transducers include one-dimensional arrays of ultrasonic transducers. In other embodiments, the subsets of ultrasonic transducers include two sub-apertures of a two-dimensional array of ultrasonic transducers. In some embodiments, the ultrasonic signals transmitted from the first subset of ultrasonic transducers and the second subset of ultrasonic transducers utilize a same frequency. The artery motion reconstructed from each subsets of ultrasonic transducers is then analyzed to extract the pulse transit time (PTT) from one subset of ultrasonic transducers to another and estimate of the pulse wave velocity (PWV) is derived as PWV=L/PTT, where L is the distance between the two transducers.

The described embodiments enable loose alignment by the user of the subsets of ultrasonic transducers. For example, in one embodiment there are two linear arrays ultrasonic transducers located transversally at two arterial sites at a distance L. By scanning the field of view of an array, the artery motion can be identified with the proper electronic scan and signal processing pipeline with a much tighter resolution compared to the manual alignment, that requires only to have the vessel within the array field of view (FoV). In some embodiments, estimating the pulse transit time (PTT) from the pulsatile motion extracted on each site can be, to mention a few, cross-correlation of the curves or derived waveforms, time delays between fiducial markers identified on these or more complex AI/ML algorithms.

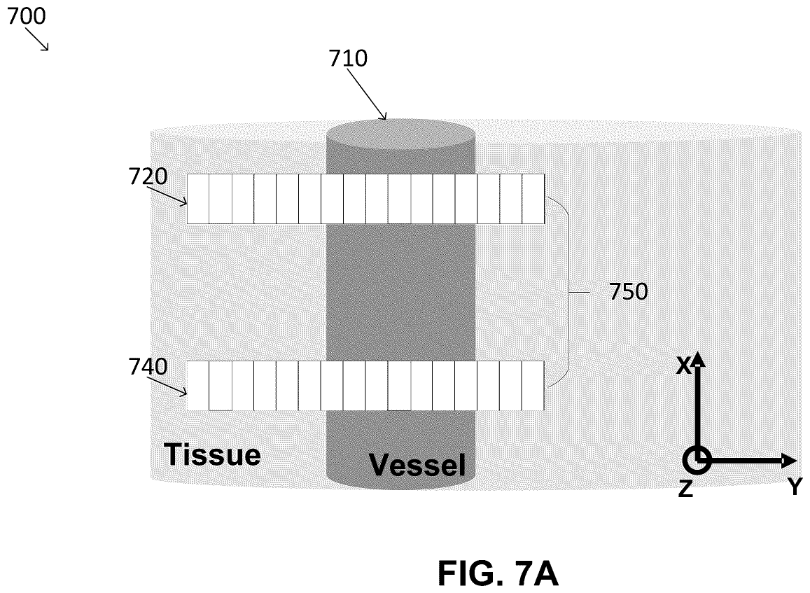

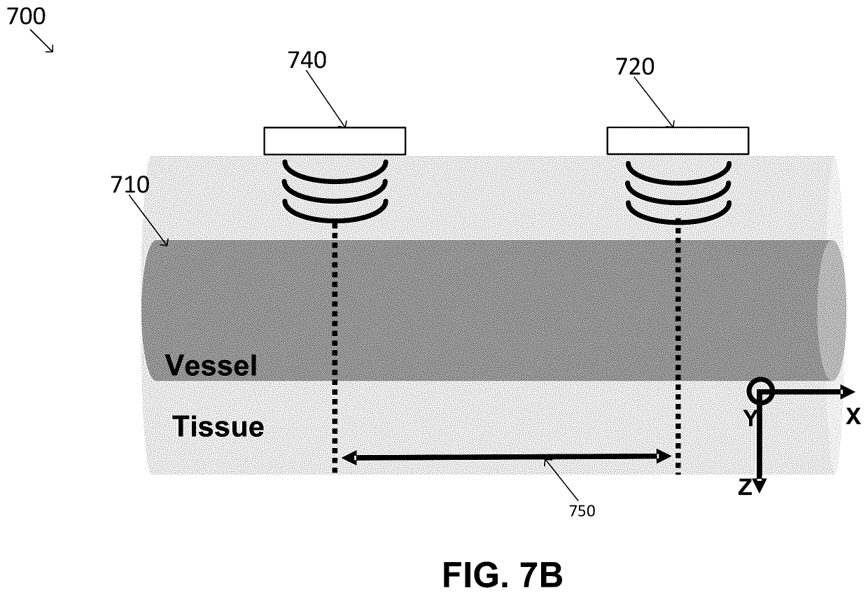

FIGS. 7A and 7B illustrate different views of a graphical representation of an ultrasonic sensing system 700 including two linear ultrasonic transducers arrays 720 and 740 for performing cardiovascular monitoring, according to some embodiments. FIG. 7A illustrates a top view of an ultrasonic sensing system including two linear ultrasonic transducers arrays 720 and 740 for performing cardiovascular monitoring, and FIG. 7B illustrates a side view of the same. Linear ultrasonic transducers arrays 720 and 740 are positioned transversally to blood vessel 710 and are separated by known distance 750 (e.g., L as described above).

The hardware controller is configured to control transmission of ultrasonic signals from linear ultrasonic transducers arrays 720 and 740 and configured to control receipt of reflected ultrasonic signals at linear ultrasonic transducers arrays 720 and 740, wherein the reflected ultrasonic signals sense movement of at least one wall of the blood vessel. As utilized herein, the top wall refers to the blood vessel wall closest to the ultrasonic transducer arrays and the bottom wall refers to the blood vessel wall farthest from the ultrasonic transducer arrays. The top and bottom walls might display different dynamics and both can be used for cardiovascular monitoring. It should be appreciated that layers of the blood vessel walls (e.g., intima, media, adventitia) can also be used. In some embodiments, the reflected ultrasonic signals received ultrasonic transducer array 720 are received at the digital processing module using a first receive channel and the reflected ultrasonic signals received at ultrasonic transducer array 740 are received at the digital processing module using a second receive channel. In some embodiments, the reflected ultrasonic signals are received at the digital processing module from the first receive channel and the second receive channel. In some embodiments, the digital signal processing module is configured to account for the phase shift between a plurality of instances of the reflected ultrasonic signals received from the first receive channel and to account for the phase shift between a plurality of instances of the reflected ultrasonic signals received from the second receive channel. The artery motion is reconstructed (e.g., at processing unit 130) from ultrasonic transducers arrays 720 and 740 and it is analyzed to extract the pulse transit time (PTT) from one subset of ultrasonic transducers to another and estimate of the pulse wave velocity (PWV) is derived as PWV=L/PTT, since the distance between the two transducers is known.

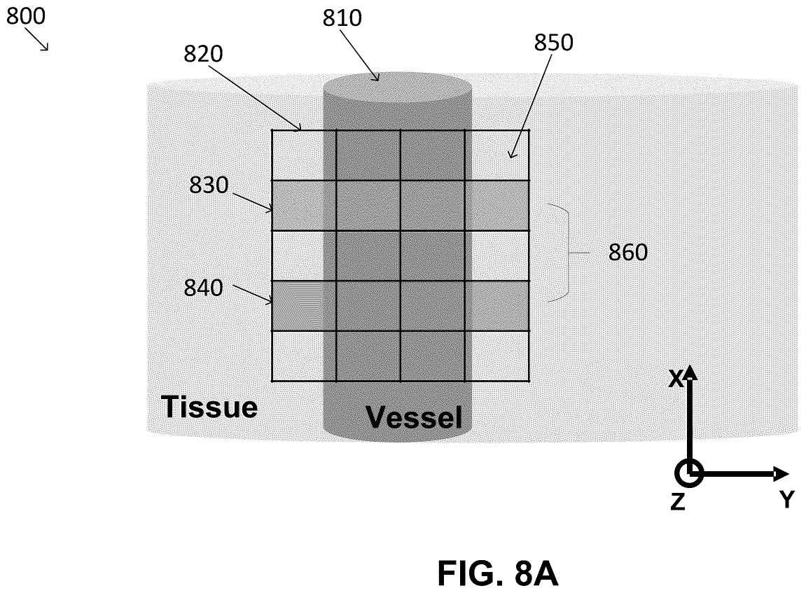

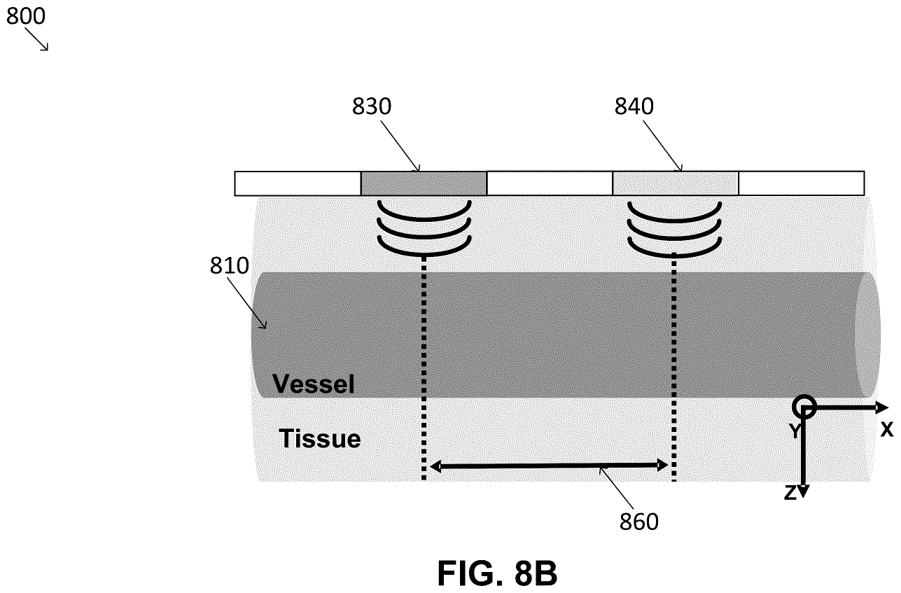

FIGS. 8A and 8B illustrate different views of a graphical representation of an ultrasonic sensing system 800 including a two-dimensional ultrasonic transducer array 820 of for performing cardiovascular monitoring, according to some embodiments. FIG. 8A illustrates a top view of an ultrasonic sensing system including a two-dimensional ultrasonic transducer array 820 of ultrasonic transducers 850 for performing cardiovascular monitoring, and FIG. 8B illustrates a side view of the same. During operation, sub-apertures 830 and 840 of two-dimensional ultrasonic transducer array 820 are activated for performing cardiovascular monitoring, while other transducers of two-dimensional ultrasonic transducer array 820 are not activated. Sub-apertures 830 and 840 are positioned transversally to blood vessel 810 and are separated by known distance 860 (e.g., L as described above), which is twice the elevation of one ultrasonic transducer 850 in FIGS. 8A and 8B.

The hardware controller is configured to control transmission of ultrasonic signals from sub-apertures 830 and 840 of two-dimensional ultrasonic transducer array 820 and configured to control receipt of reflected ultrasonic signals at sub-apertures 830 and 840 of two-dimensional ultrasonic transducer array 820, wherein the reflected ultrasonic signals sense movement of a wall of the blood vessel. As utilized herein, the top wall refers to the blood vessel wall closest to the ultrasonic transducer arrays and the bottom wall refers to the blood vessel wall farthest from the ultrasonic transducer arrays. The top and bottom walls might display different dynamics and both can be used for cardiovascular monitoring. It should be appreciated that layers of the blood vessel walls (e.g., intima, media, adventitia) can also be used. In some embodiments, the reflected ultrasonic signals received at sub-aperture 830 are received at the digital processing module using a first receive channel and the reflected ultrasonic signals received at sub-aperture 830 are received at the digital processing module using a second receive channel. In some embodiments, the reflected ultrasonic signals are received at the digital processing module from the first receive channel and the second receive channel. In some embodiments, the digital signal processing module is configured to account for the phase shift between a plurality of instances of the reflected ultrasonic signals received from the first receive channel and to account for the phase shift between a plurality of instances of the reflected ultrasonic signals received from the second receive channel. The artery motion is reconstructed (e.g., at processing unit 130) from sub-apertures 830 and 840 is analyzed to extract the pulse transit time (PTT) from one subset of ultrasonic transducers to another and estimate of the pulse wave velocity (PWV) is derived as PWV=L/PTT, since the distance between the two transducers is known.

### Example Automatic Alignment of a Traversal Array of Ultrasonic Transducers

Embodiments described herein enable continuous cardiovascular monitoring using a wearable system for ultrasonic sensing of blood vessels. In order to provide such cardiovascular monitoring, the system described herein enables automatic alignment of ultrasonic sensors with the target blood vessel. The embodiments described herein provide for automatic alignment with underlying blood vessels by determining which ultrasonic transducer of an array of ultrasonic transducers placed transversely to the target blood vessel exhibits alignment with the target blood vessel. The described embodiments perform automatic alignment without the need for a human operator and/or without image analysis, allowing for a wearable system capable of cardiovascular monitoring to perform the described alignment automatically. It should be appreciated that the described embodiments can also be applied to situations where the array of ultrasonic transducers is placed with an in-plane or out-of-plane angle with the target blood vessel.

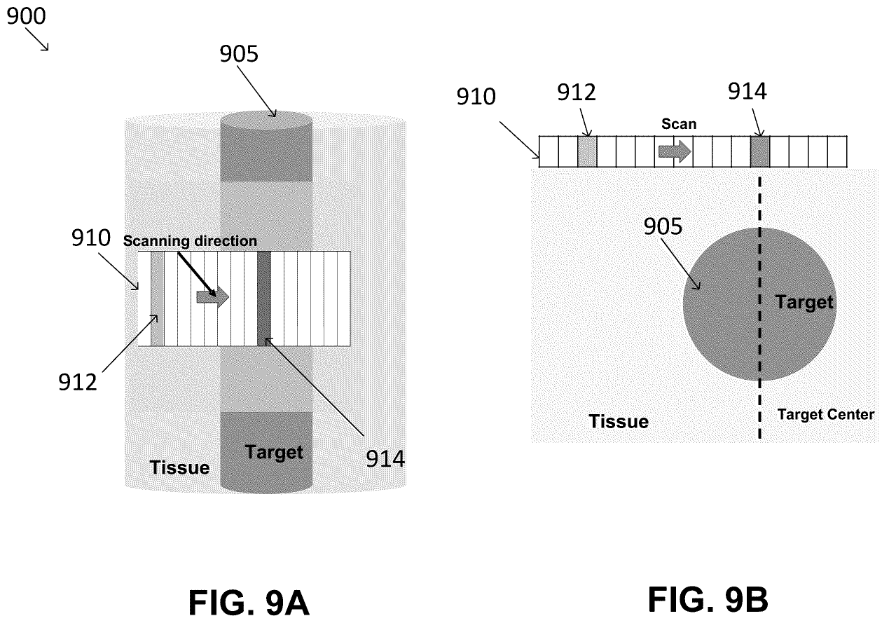

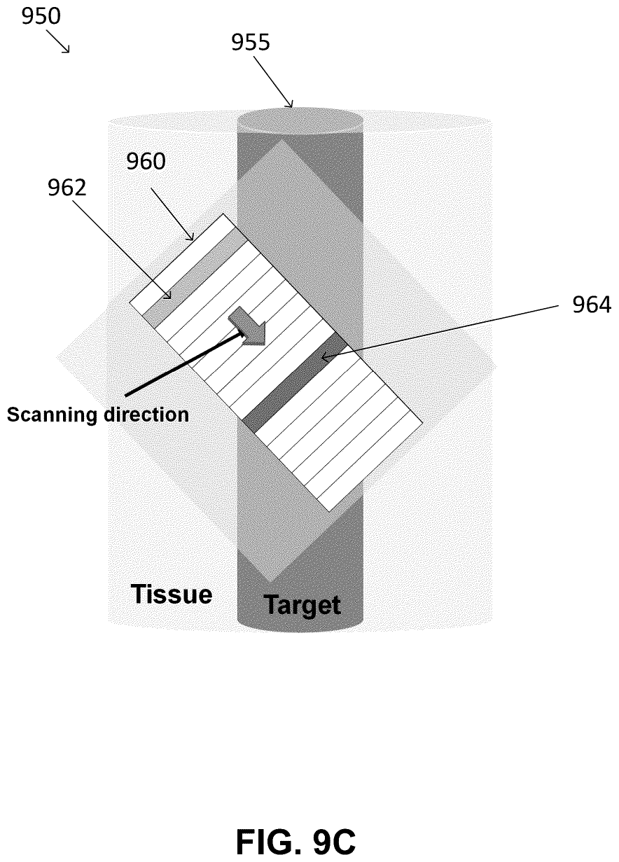

FIGS. 9A, 9B, and 9C illustrate different views of graphical representations of automatic alignment of a transversal array of ultrasonic transducers, according to some embodiments. FIG. 9A illustrates a top view of automatic alignment operation 900, where FIG. 9B illustrates a cross-section view of the same. During automatic alignment operation 900, ultrasonic transducer array 910 is placed on a human body transversally to target blood vessel 905. Ultrasonic transducer array 910 is a linear array of ultrasonic transducers, of which ultrasonic transducer 912 is a representative ultrasonic transducer.

A plurality of instances of an ultrasonic scanning operation directed towards target blood vessel 905 using ultrasonic transducer array 910 is performed in a scanning direction, as indicated in FIGS. 9A and 9B. In some embodiments, each ultrasonic transducer is activated independently during an instance of the ultrasonic scanning operation. In other embodiments, subsets of ultrasonic transducers are activated during an instance of the ultrasonic scanning operation (e.g., using beamforming). An instance of the ultrasonic scanning operation generates an ultrasonic signal toward target blood vessel 905 and receives at least one reflected ultrasonic signal from target blood vessel 905 at a receiving ultrasonic transducer. In accordance with various embodiments, a signal amplitude and/or a time of flight are determined for each of the at least one reflected ultrasonic signal of each instance of the ultrasonic scanning operation. In other embodiments, other properties or characteristics are determined, such as a phase shift or a Doppler signal. The signal amplitude and/or a time of flight are analyzed to determine which ultrasonic scanning operation, and thus which receiving ultrasonic transducer, exhibits alignment with target blood vessel 905.

As illustrated in FIGS. 9A and 9B, ultrasonic transducer 914 of ultrasonic transducer array 910 is the ultrasonic transducer exhibiting alignment with target blood vessel 905. As shown, ultrasonic transducer 914 of ultrasonic transducer array 910 is most closely aligned with the center of target blood vessel 905.

It should be appreciated that an ultrasonic sensor performing the described automatic alignment can be placed in any transversal location relative to the underlying target blood vessel other than exactly parallel to the target blood vessel. Automatic alignment operation 900 of FIGS. 9A and 9B illustrates ultrasonic transducer array 910 as being substantially perpendicular to target blood vessel 905. It should be appreciated that FIGS. 9A and 9B are generalizations of an automatic alignment operation so as to illustrate the described embodiments without obfuscation, and that in practice a blood vessel might have variations in size and shape. In general, the automatic alignment operation described herein is operable so long as the target blood vessel is in the field of view of ultrasonic transducer array and is positioned transversally to the target blood vessel.

To further illustrate the automatic alignment operation described herein, FIG. 9C illustrates a top view of automatic alignment operation 950, where ultrasonic transducer array 960 is placed on a human body transversally and at a non-perpendicular angle to target blood vessel 955. Ultrasonic transducer array 960 is a linear array of ultrasonic transducers, of which ultrasonic transducer 962 is a representative ultrasonic transducer.

A plurality of instances of an ultrasonic scanning operation directed towards target blood vessel 955 using ultrasonic transducer array 960 is performed in a scanning direction, as indicated in FIG. 9C. In some embodiments, each ultrasonic transducer is activated independently during an instance of the ultrasonic scanning operation. In other embodiments, subsets of ultrasonic transducers are activated during an instance of the ultrasonic scanning operation (e.g., using beamforming). An instance of the ultrasonic scanning operation generates an ultrasonic signal toward target blood vessel 955 and receives at least one reflected ultrasonic signal from target blood vessel 955 at a receiving ultrasonic transducer. In accordance with various embodiments, a signal amplitude and/or a time of flight are determined for each of the at least one reflected ultrasonic signal of each instance of the ultrasonic scanning operation. The signal amplitude and/or a time of flight are analyzed to determine which ultrasonic scanning operation, and thus which receiving ultrasonic transducer, exhibits alignment with target blood vessel 955. In other embodiments, other properties or characteristics are determined, such as a phase shift or a Doppler signal. As illustrated in FIG. 9C, ultrasonic transducer 964 of ultrasonic transducer array 960 is the ultrasonic transducer exhibiting alignment with target blood vessel 955. As shown, ultrasonic transducer 964 of ultrasonic transducer array 960 is most closely aligned with the center of target blood vessel 955.

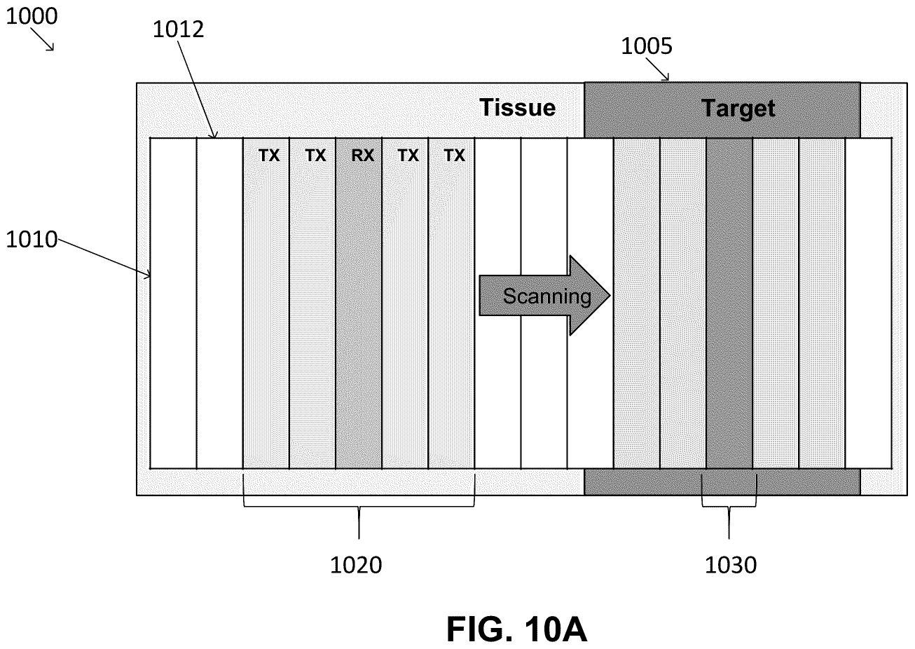

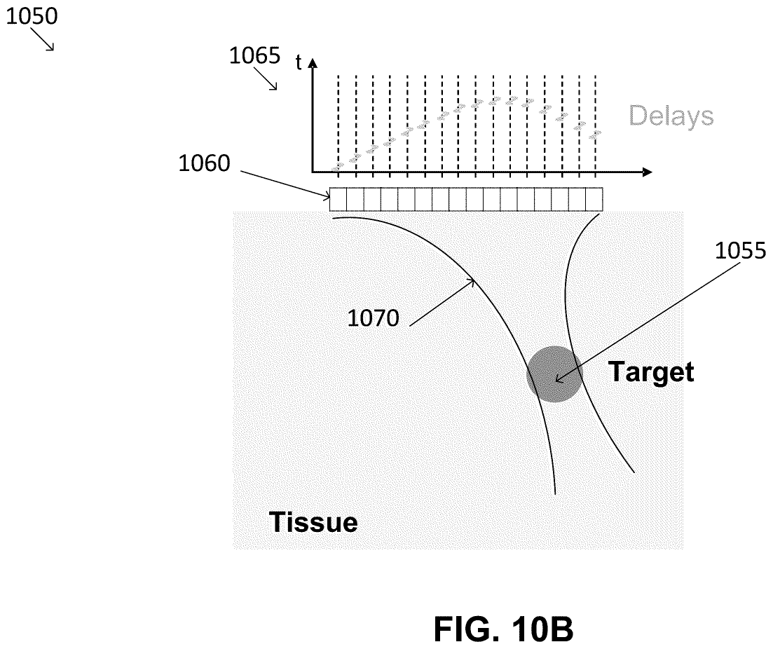

FIGS. 10A and 10B illustrate different views of graphical representations of beamforming at a transversal array of ultrasonic transducers, according to some embodiments. Embodiments described herein utilize beamforming at an ultrasonic transducer array to perform ultrasonic scanning operations of an automatic alignment operation. FIG. 10A illustrates an example instance of an ultrasonic scanning operation 1000 using beamforming at ultrasonic transducer array 1010. As illustrated in FIG. 10A, a subset 1020 of ultrasonic transducers 1012 is utilized during each instance of the ultrasonic scanning operation, where subset 1020 is scanned across ultrasonic transducer array 1010 in successive instances of the ultrasonic scanning operation. As illustrated, subset 1020 includes five ultrasonic transducers, four of which are used to perform beamforming of a transmitted ultrasonic signal (Tx), with the center ultrasonic transducer (Rx) receiving the reflected ultrasonic signal.

FIG. 10B illustrates an example beamforming operation 1050 illustrating how relative delayed activation of ultrasonic transducers of an ultrasonic sensing array 1060 effectuates the beamforming. Graph 1065 illustrates example relative time delays for activation of ultrasonic transducers of ultrasonic sensing array 1060. By delaying activation of ultrasonic transducers relative to other ultrasonic transducers of ultrasonic sensing array 1060, the generated ultrasonic beam 1070 can be steered to a desired location within the human body (tissue), e.g., target blood vessel 1055. In this manner, the described embodiments can utilize beamforming and/or beam steering to performing ultrasonic scanning operations. It should be appreciated that FIG. 10B is an example of a beamforming operation, and that any number of ultrasonic transducers can be used to perform beamforming, where symmetric activation delays of ultrasonic transducers of the activated ultrasonic transducers will generate an ultrasonic beam centered over a middle ultrasonic transducer and asymmetric activation delays of ultrasonic transducers will generate an ultrasonic beam steered towards a location away from the middle ultrasonic transducer of the activated ultrasonic transducers.

With reference to FIG. 10A, an instance of the ultrasonic scanning operation generates an ultrasonic signal toward target blood vessel 1005 and receives at least one reflected ultrasonic signal from target blood vessel 1005 at a receiving ultrasonic transducer (Rx). In accordance with various embodiments, a signal amplitude and/or a time of flight are determined for each of the at least one reflected ultrasonic signal of each instance of the ultrasonic scanning operation. In other embodiments, other properties or characteristics are determined, such as a phase shift or a Doppler signal. The signal amplitude and/or a time of flight are analyzed to determine which ultrasonic scanning operation, and thus which receiving ultrasonic transducer, exhibits alignment with target blood vessel 1005. As illustrated in FIG. 10A ultrasonic transducer 1030 of ultrasonic transducer array 1010 is the ultrasonic transducer exhibiting alignment with target blood vessel 1005.

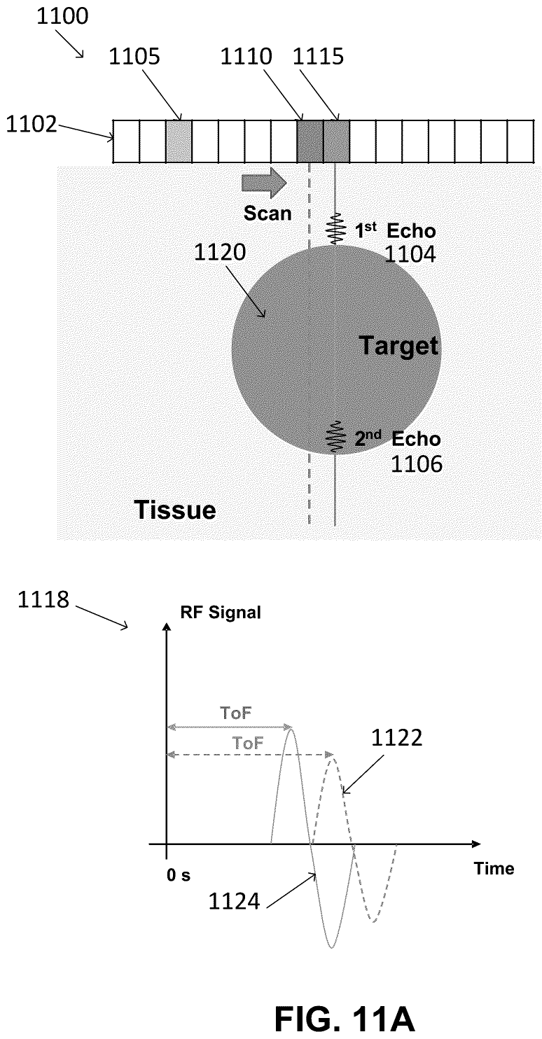

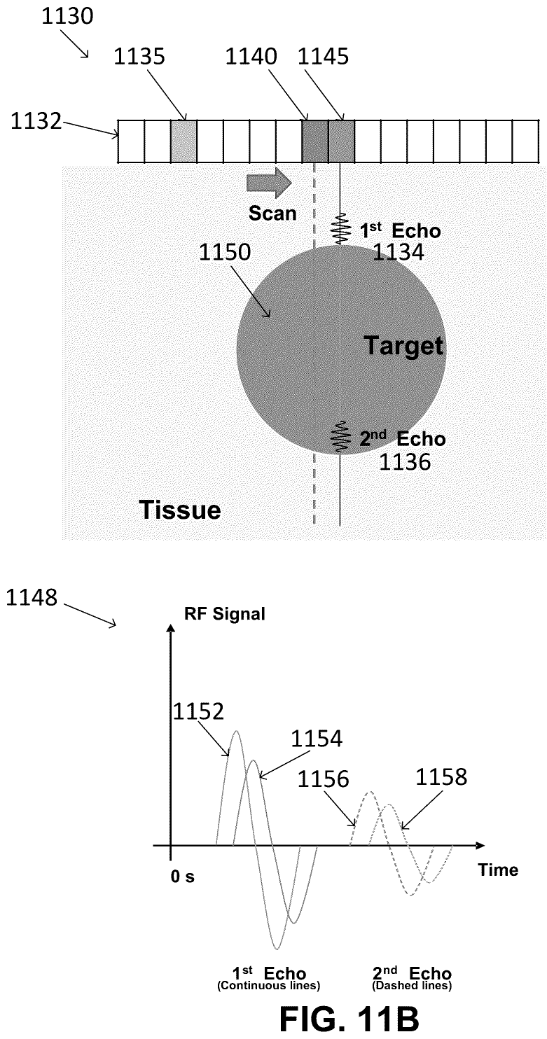

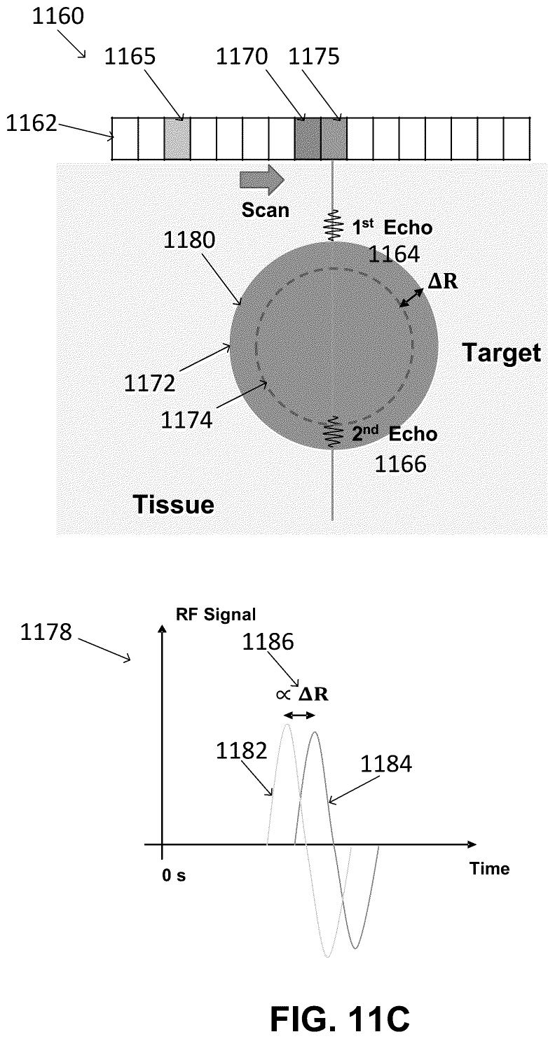

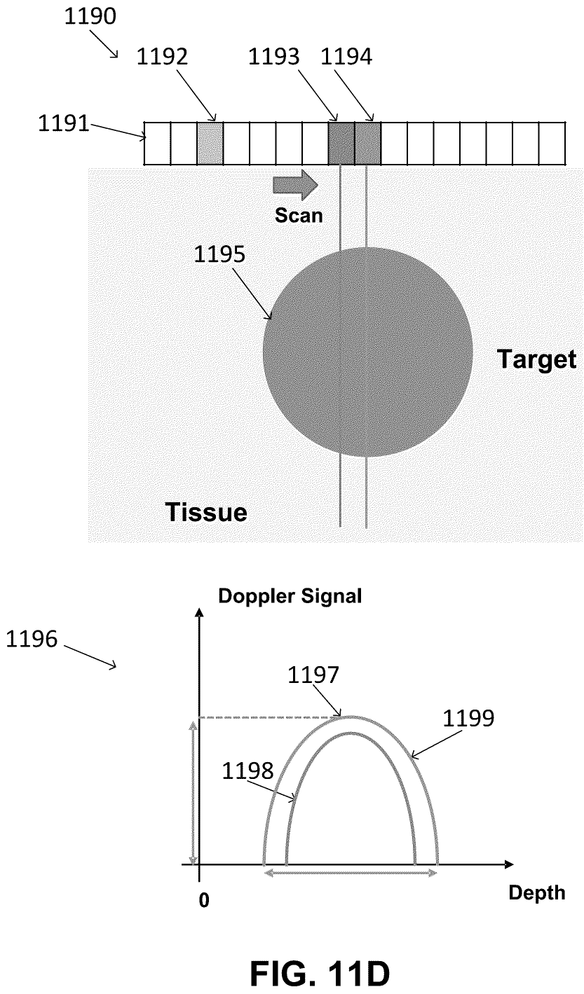

FIGS. 11A, 11B, 11C, and 11D illustrate different embodiments of performing automatic alignment of a transversal array, according to embodiments. FIG. 11A illustrates automatic alignment operation 1100, in which the amplitude and/or time of flight (ToF) of the reflected ultrasonic signal received at ultrasonic transducers of ultrasonic transducer array 1102 is used for determining which ultrasonic transducers of ultrasonic transducer array 1102 exhibits alignment with target blood vessel 1120. A plurality of instances of an ultrasonic scanning operation directed towards target blood vessel 1120 using ultrasonic transducer array 1102 is performed in a scanning direction, as indicated in FIG. 11A. In some embodiments, each ultrasonic transducer of ultrasonic transducer array 1102 (e.g., ultrasonic transducer 1105) is activated independently during an instance of the ultrasonic scanning operation. In other embodiments, subsets of ultrasonic transducers of ultrasonic transducer array 1102 are activated during an instance of the ultrasonic scanning operation (e.g., using beamforming). An instance of the ultrasonic scanning operation generates an ultrasonic signal toward target blood vessel 1120 and receives at least one reflected ultrasonic signal (e.g., an echo) from target blood vessel 1120 at a receiving ultrasonic transducer. During each instance of the ultrasonic scanning operation that transmits towards target blood vessel 1120, two reflected ultrasonic signals may be received, e.g., first echo 1104 and second echo 1106, where first echo 1104 is the reflected ultrasonic signal from the closest wall of target blood vessel 1120 and second echo 1106 is the reflected ultrasonic signal from the furthest wall of target blood vessel 1120.

In one embodiment, the signal amplitude of first echo 1104 is determined and used for determining which ultrasonic transducer of ultrasonic transducer array 1102 exhibits best alignment with target blood vessel 1120. The ultrasonic transducer of the array of ultrasonic transducers selected as exhibiting alignment with the target blood vessel 1120 exhibits the largest signal amplitude of the signal amplitudes of each instance of the ultrasonic scanning operation. For example, FIG. 11A illustrates graph 1118 which shows the signal amplitude over time for ultrasonic transducer 1110 (shown at curve 1122) and ultrasonic transducer 1115 (shown at curve 1124). As shown in graph 1118, the signal amplitude of the first echo 1104 of ultrasonic transducer 1115 is greater than the signal amplitude of the first echo 1104 of ultrasonic transducer 1110. In the illustrated embodiment, ultrasonic transducer 1115 is selected as the ultrasonic transducer exhibiting alignment with the target blood vessel 1120, as the signal amplitude of the first echo 1104 of ultrasonic transducer 1115 is largest.

In another embodiment, the signal amplitude of second echo 1106 is determined and used for determining which ultrasonic transducer of ultrasonic transducer array 1102 exhibits best alignment with target blood vessel 1120. The ultrasonic transducer of the array of ultrasonic transducers selected as exhibiting alignment with the target blood vessel 1120 exhibits the smallest signal amplitude of the signal amplitudes of each instance of the ultrasonic scanning operation. For example, the signal amplitude of the second echo 1106 of ultrasonic transducer 1115 is less than the signal amplitude of the second echo 1106 of ultrasonic transducer 1110. In the illustrated embodiment, ultrasonic transducer 1115 is selected as the ultrasonic transducer exhibiting alignment with the target blood vessel 1120, as the signal amplitude of the second echo 1106 of ultrasonic transducer 1115 is largest.

In another embodiment, the time of flight of first echo 1104 is determined and used for determining which ultrasonic transducer of ultrasonic transducer array 1102 exhibits best alignment with target blood vessel 1120. The ultrasonic transducer of the array of ultrasonic transducers selected as exhibiting alignment with the target blood vessel 1120 exhibits the smallest time of flight of each instance of the ultrasonic scanning operation. For example, as shown in graph 1118, the time of flight of the first echo 1104 of ultrasonic transducer 1115 is less than the time of flight of the first echo 1104 of ultrasonic transducer 1110. In the illustrated embodiment, ultrasonic transducer 1115 is selected as the ultrasonic transducer exhibiting alignment with the target blood vessel 1120, as the time of flight of the first echo 1104 of ultrasonic transducer 1115 is smallest.

In another embodiment, the time of flight of second echo 1106 is determined and used for determining which ultrasonic transducer of ultrasonic transducer array 1102 exhibits best alignment with target blood vessel 1120. The ultrasonic transducer of the array of ultrasonic transducers selected as exhibiting alignment with the target blood vessel 1120 exhibits the largest time of flight of each instance of the ultrasonic scanning operation. For example, the time of flight of the second echo 1106 of ultrasonic transducer 1115 is greater than the time of flight of the second echo 1106 of ultrasonic transducer 1110. In the illustrated embodiment, ultrasonic transducer 1115 is selected as the ultrasonic transducer exhibiting alignment with the target blood vessel 1120, as the time of flight of the second echo 1106 of ultrasonic transducer 1115 is largest.

FIG. 11B illustrates automatic alignment operation 1130, in which the time of flight differential and/or the signal amplitude differential between two reflected ultrasonic signals received at ultrasonic transducers of ultrasonic transducer array 1132 for the same instance of a signal transmission is used for determining which ultrasonic transducers of ultrasonic transducer array 1132 exhibits alignment with target blood vessel 1150. The time of flight differential and the signal amplitude differential between two reflected ultrasonic signals (e.g., a first echo 1134 and second echo 1136) for the same instance of a signal transmission is indicative of the relative thickness of target blood vessel 1150, where the largest time of flight differential and large signal amplitude differential is indicative of the widest portion of target blood vessel 1150, which is the location of the ultrasonic sensor exhibiting best alignment with target blood vessel 1150.

A plurality of instances of an ultrasonic scanning operation directed towards target blood vessel 1150 using ultrasonic transducer array 1132 is performed in a scanning direction, as indicated in FIG. 11B. In some embodiments, each ultrasonic transducer of ultrasonic transducer array 1132 (e.g., ultrasonic transducer 1135) is activated independently during an instance of the ultrasonic scanning operation. In other embodiments, subsets of ultrasonic transducers of ultrasonic transducer array 1132 are activated during an instance of the ultrasonic scanning operation (e.g., using beamforming). An instance of the ultrasonic scanning operation generates an ultrasonic signal toward target blood vessel 1150 and receives at least one reflected ultrasonic signal (e.g., an echo) from target blood vessel 1150 at a receiving ultrasonic transducer. During each instance of the ultrasonic scanning operation that transmits towards target blood vessel 1150, two reflected ultrasonic signals may be received, e.g., first echo 1134 and second echo 1136, where first echo 1134 is the reflected ultrasonic signal from the closest wall of target blood vessel 1150 and second echo 1136 is the reflected ultrasonic signal from the furthest wall of target blood vessel 1150.

In one embodiment, the signal amplitude of first echo 1134 and the signal amplitude of second echo 1136 is determined and used for determining which ultrasonic transducer of ultrasonic transducer array 1132 exhibits best alignment with target blood vessel 1150. The ultrasonic transducer of the array of ultrasonic transducers selected as exhibiting alignment with the target blood vessel 1150 exhibits the largest signal amplitude differential of the signal amplitude differentials of each instance of the ultrasonic scanning operation. For example, FIG. 11B illustrates graph 1148 which shows the signal amplitude over time for ultrasonic transducer 1145 (first echo 1134 shown at curve 1152 and second echo 1136 shown at curve 1158) and ultrasonic transducer 1140 (first echo 1134 shown at curve 1154 and second echo 1136 shown at curve 1156). As shown in graph 1148, the signal amplitude differential between first echo 1134 and second echo 1136 for ultrasonic transducer 1145 is greater than the signal amplitude differential between first echo 1134 and second echo 1136 for ultrasonic transducer 1140. In the illustrated embodiment, ultrasonic transducer 1145 is selected as the ultrasonic transducer exhibiting alignment with the target blood vessel 1150, as the signal amplitude differential between of first echo 1134 and second echo 1136 of ultrasonic transducer 1145 is largest.

In another embodiment, the time of flight of first echo 1134 and the time of flight of second echo 1136 is determined and used for determining which ultrasonic transducer of ultrasonic transducer array 1132 exhibits best alignment with target blood vessel 1150. The ultrasonic transducer of the array of ultrasonic transducers selected as exhibiting alignment with the target blood vessel 1120 exhibits the largest time of flight differential of the time of flight differentials of each instance of the ultrasonic scanning operation. For example, as shown in graph 1148, the time of flight differential between first echo 1134 and second echo 1136 for ultrasonic transducer 1145 is greater than the time of flight differential between first echo 1134 and second echo 1136 for ultrasonic transducer 1140. In the illustrated embodiment, ultrasonic transducer 1145 is selected as the ultrasonic transducer exhibiting alignment with the target blood vessel 1150, as the time of flight differential between of first echo 1134 and second echo 1136 of ultrasonic transducer 1145 is largest.

FIG. 11C illustrates automatic alignment operation 1160, in which dynamic parameter detection is used for determining which ultrasonic transducers of ultrasonic transducer array 1162 exhibits alignment with target blood vessel 1180. Automatic alignment operation 1160 performs two instances of the ultrasonic scanning operation at each ultrasonic transducer of ultrasonic transducer array 1162, and the ultrasonic transducer of ultrasonic transducer array 1162 selected as exhibiting alignment with target blood vessel 1180 exhibits the largest time of flight differential between the reflected ultrasonic signals of the two instances at the ultrasonic transducer. The time of flight differential is indicative of and proportional to the radius variation of the target blood vessel 1180 during successive reflected signal acquisitions for an ultrasonic transducer, where the largest radius variation is indicative of the widest portion of target blood vessel 1180, which is the location of the ultrasonic sensor exhibiting best alignment with target blood vessel 1180.

A plurality of instances of an ultrasonic scanning operation directed towards target blood vessel 1180 using ultrasonic transducer array 1162 is performed in a scanning direction, as indicated in FIG. 11C, where each ultrasonic transducer performs two ultrasonic sensing operations. In some embodiments, each ultrasonic transducer of ultrasonic transducer array 1162 (e.g., ultrasonic transducer 1165 and ultrasonic transducer 1175) is activated independently during an instance of the ultrasonic scanning operation. In other embodiments, subsets of ultrasonic transducers of ultrasonic transducer array 1162 are activated during an instance of the ultrasonic scanning operation (e.g., using beamforming). An instance of the ultrasonic scanning operation generates an ultrasonic signal toward target blood vessel 1180 and receives at least one reflected ultrasonic signal (e.g., an echo) from target blood vessel 1180 at a receiving ultrasonic transducer. The ultrasonic sensing operation is performed twice at each ultrasonic transducer, shown as operation 1172 and operation 1174, where operation 1174 illustrates a contraction of blood vessel 1180 relative to operation 1172. During each instance of the ultrasonic scanning operation that transmits towards target blood vessel 1180, two reflected ultrasonic signals may be received, e.g., first echo 1164 and second echo 1166, where first echo 1164 is the reflected ultrasonic signal from the closest wall of target blood vessel 1180 and second echo 1166 is the reflected ultrasonic signal from the furthest wall of target blood vessel 1180.

In one embodiment, the signal amplitude of first echo 1164 is determined twice for each ultrasonic transducer of ultrasonic transducer array 1162 and used for determining which ultrasonic transducer of ultrasonic transducer array 1162 exhibits best alignment with target blood vessel 1180. The ultrasonic transducer of ultrasonic transducer array 1162 selected as exhibiting alignment with the target blood vessel 1180 exhibits the smallest signal amplitude differential between the two instances of the ultrasonic scanning operation performed at each ultrasonic transducer. For example, FIG. 11C illustrates graph 1178 which shows the signal amplitude over time for ultrasonic transducer 1175 (first echo 1164 shown at curve 1182 and second echo 1166 shown at curve 1184). The signal amplitude differential 1186 is determined for the two ultrasonic scanning operations performed at each ultrasonic transducer. In the illustrated embodiment, ultrasonic transducer 1175 is selected as the ultrasonic transducer exhibiting alignment with the target blood vessel 1180, as the signal amplitude differential the two instances of the ultrasonic scanning operations performed at each ultrasonic transducer is smallest.

In another embodiment, the time of flight of first echo 1164 is determined twice for each ultrasonic transducer of ultrasonic transducer array 1162 and used for determining which ultrasonic transducer of ultrasonic transducer array 1162 exhibits best alignment with target blood vessel 1180. The ultrasonic transducer of ultrasonic transducer array 1162 selected as exhibiting alignment with the target blood vessel 1180 exhibits the largest time of flight differential between the two instances of the ultrasonic scanning operation performed at each ultrasonic transducer. The time of flight differential is determined for the two ultrasonic scanning operations performed at each ultrasonic transducer. In the illustrated embodiment, ultrasonic transducer 1175 is selected as the ultrasonic transducer exhibiting alignment with the target blood vessel 1180, as the time of flight differential the two instances of the ultrasonic scanning operations performed at each ultrasonic transducer is largest.

FIG. 11D illustrates automatic alignment operation 1190, in which Doppler signal detection is used for determining which ultrasonic transducers of ultrasonic transducer array 1191 exhibits alignment with target blood vessel 1195. Automatic alignment operation 1190 performs multiple instances of the ultrasonic scanning operation at each ultrasonic transducer of ultrasonic transducer array 1191, and the ultrasonic transducer of ultrasonic transducer array 1191 selected as exhibiting alignment with target blood vessel 1195 exhibits the largest width of the velocity profile and/or the largest velocity amplitude of the blood within target blood vessel 1195. The velocity profile (e.g., blood vessel thickness at the point of measurement) and/or the velocity amplitude (where velocity of blood flow is largest in the middle of target blood vessel 1195) are indicative of and proportional to relative alignment of an ultrasonic transducer of ultrasonic transducer array 1191. The largest width of the velocity profile and the largest velocity amplitude are indicative of the widest portion of target blood vessel 1195, which is the location of the ultrasonic sensor exhibiting best alignment with target blood vessel 1195.

A plurality of instances of an ultrasonic scanning operation directed towards target blood vessel 1195 using ultrasonic transducer array 1191 is performed in a scanning direction, as indicated in FIG. 11D, where each ultrasonic transducer performs multiple ultrasonic sensing operations. In some embodiments, each ultrasonic transducer of ultrasonic transducer array 1191 (e.g., ultrasonic transducer 1192) is activated independently during an instance of the ultrasonic scanning operation. In other embodiments, subsets of ultrasonic transducers of ultrasonic transducer array 1191 are activated during an instance of the ultrasonic scanning operation (e.g., using beamforming). An instance of the ultrasonic scanning operation generates an ultrasonic signal toward target blood vessel 1195 and receives at least one reflected ultrasonic signal (e.g., an echo) from target blood vessel 1195 at a receiving ultrasonic transducer. The ultrasonic sensing operation is performed multiple times at each ultrasonic transducer to determine the Doppler signal at each ultrasonic transducer. During each instance of the ultrasonic scanning operation that transmits towards target blood vessel 1195, multiple reflected ultrasonic signals are received to determine the maximum flow rate at each position by comparing the reflected ultrasonic signals.

The ultrasonic transducer of ultrasonic transducer array 1191 selected as exhibiting alignment with the target blood vessel 1195 exhibits the largest width of the velocity profile and/or the largest velocity amplitude of the blood within target blood vessel 1195. For example, FIG. 11C illustrates graph 1196 which shows the Doppler signal amplitude over blood vessel width for ultrasonic transducer 1193 (curve 1198) and ultrasonic transducer 1194 (curve 1199). The Doppler signal amplitude and blood vessel depth (e.g., thickness) is determined for each ultrasonic transducer. In the illustrated embodiment, ultrasonic transducer 1194 is selected as the ultrasonic transducer exhibiting alignment with the target blood vessel 1195, as the Doppler signal amplitude 1197 and blood vessel width are largest.

It should be appreciated that while embodiments described herein utilize signal amplitude, time of flight, and phase shift to select an ultrasonic transducer exhibiting alignment with a target blood vessel, that other properties or characteristics can be used in accordance with the described embodiments. For example, in some embodiments, contrast of a signal amplitude can be used for selecting an ultrasonic transducer exhibiting alignment with a target blood vessel. Contrast is a derivative of signal amplitude, where the walls of the target blood vessel appear bright in contrast (e.g., white), while the vessel lumen (where the blood flows) appears dark in contrast (e.g., black). The largest distance can be determined using the distance at the point of maximum contrast, also indicating a midpoint of the target blood vessel, where the ultrasonic transducer at the point of largest distance exhibit alignment with a target blood vessel.

### Example Operations for Automatic Alignment of a Transversal Array of Ultrasonic Transducers

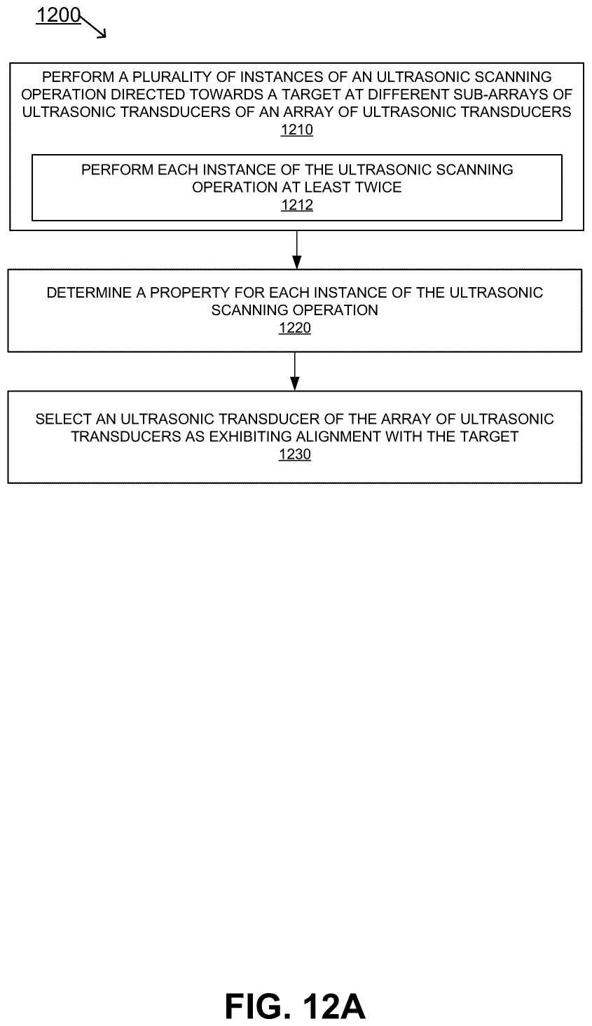

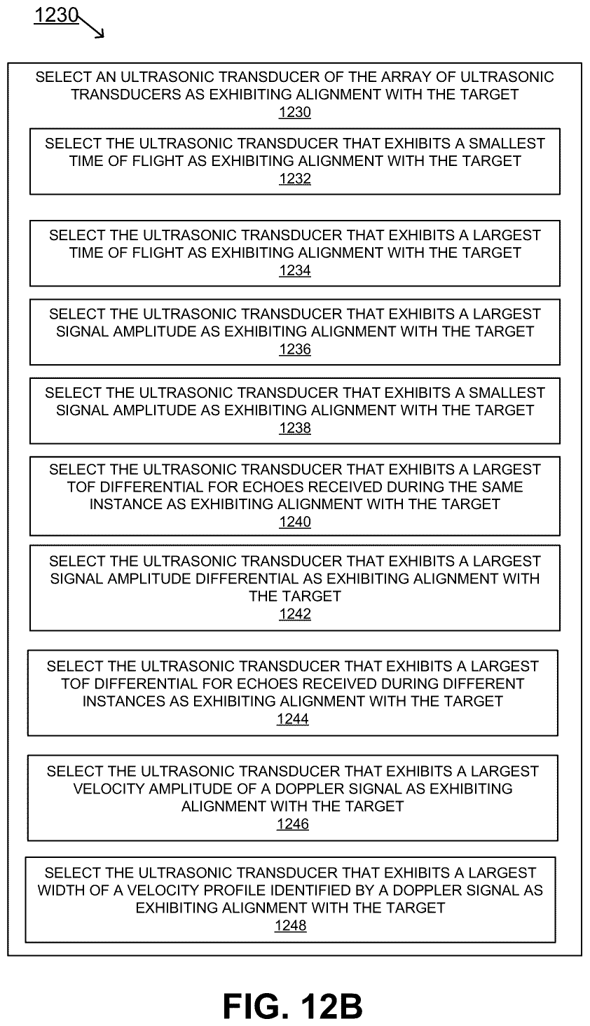

FIGS. 12A and 12B illustrate a flow diagram of an example method for automatic alignment of a transversal array of ultrasonic transducers, according to various embodiments. Procedures of these methods will be described with reference to elements and/or components of various figures described herein. It is appreciated that in some embodiments, the procedures may be performed in a different order than described, that some of the described procedures may not be performed, and/or that one or more additional procedures to those described may be performed. The flow diagrams include some procedures that, in various embodiments, are carried out by one or more processors (e.g., a host processor or a sensor processor) under the control of computer-readable and computer-executable instructions that are stored on non-transitory computer-readable storage media. It is further appreciated that one or more procedures described in the flow diagrams may be implemented in hardware, or a combination of hardware with firmware and/or software.

With reference to FIG. 12A, flow diagram 1200 illustrates an example process for automatic alignment of a transversal array of ultrasonic transducers, according to some embodiments. At procedure 1210 of flow diagram 1200, a plurality of instances of an ultrasonic scanning operation directed towards a target using an array of ultrasonic transducers is performed, wherein each instance of the plurality of instances of the ultrasonic scanning operation includes a different sub-array of ultrasonic transducers of the array of ultrasonic transducers, where a sub-array includes a portion of ultrasonic transducers of the array of ultrasonic transducers, and wherein each instance of the ultrasonic scanning operation generates an ultrasonic signal toward the target and receives at least one reflected ultrasonic signal from the target. In some embodiments, as shown at procedure 1212, each instance of the plurality of instances of the ultrasonic scanning operation is performed at least twice using each sub-array of ultrasonic transducers of the array of ultrasonic transducers, such that a reflected ultrasonic signal is received from the target twice for each sub-array. In some embodiments, a signal amplitude, a time of flight, or a phase differential between the two reflected ultrasonic signals is determined for each sub-array.

In some embodiments, the array of ultrasonic transducers is placed transversely relative to the target. In some embodiments, the sub-array of ultrasonic transducers includes one ultrasonic transducer, such that each instance of the ultrasonic scanning operation generates the ultrasonic signal at the one ultrasonic transducer and receives the at least one reflected ultrasonic signal at the one ultrasonic transducer.

In some embodiments, the sub-array of ultrasonic transducers of each instance includes a plurality of ultrasonic transducers. In some embodiments, each instance of the ultrasonic scanning operation generates the ultrasonic signal by beamforming using the plurality of ultrasonic transducers of the sub-array and receives the at least one reflected ultrasonic signal at one ultrasonic transducer of the plurality of ultrasonic transducers. In some embodiments, the beamforming is defined according to apodization and time-delay parameters of the array of ultrasonic transducers. In some embodiments, each instance of the ultrasonic scanning operation generates the ultrasonic signal by generating a plane-wave using the plurality of ultrasonic transducers of the sub-array and receives the at least one reflected ultrasonic signal at one ultrasonic transducer of the plurality of ultrasonic transducers. In some embodiments, the plane-wave generation is defined according to apodization parameters of the array of ultrasonic transducers.

At procedure 1220, at least one property is determined for each of the at least one reflected ultrasonic signal of each instance of the ultrasonic scanning operation. In some embodiments, the at least one property includes at least one of a signal amplitude, a phase shift, and a time of flight.

At procedure 1230, an ultrasonic transducer of the array of ultrasonic transducers is selected as exhibiting alignment with the target based at least in part on the signal amplitude, the phase shift, and/or the time of flight for each of the at least one reflected ultrasonic signal of each instance of the ultrasonic scanning operation. With reference to FIG. 12B, different embodiments of procedure 1230 are shown. It should be appreciated that the procedures illustrated in FIG. 12B can be utilized individually or collectively an accordance with various embodiments. For example, procedures 1232 and 1234 can both be performed to select an ultrasonic transducer exhibiting alignment with the target. In some embodiments, as shown at procedure 1232, the ultrasonic transducer of the array of ultrasonic transducers selected as exhibiting alignment with the target exhibits the smallest time of flight of the times of flight of each instance of the plurality of instances of the ultrasonic scanning operation. In some embodiments, as shown at procedure 1234, the ultrasonic transducer of the array of ultrasonic transducers selected as exhibiting alignment with the target exhibits the largest time of flight of the times of flight of each instance of the plurality of instances of the ultrasonic scanning operation. In some embodiments, as shown at procedure 1236, the ultrasonic transducer of the array of ultrasonic transducers selected as exhibiting alignment with the target exhibits the largest signal amplitude of the signal amplitudes of each instance of the ultrasonic scanning operation. In some embodiments, as shown at procedure 1238, the ultrasonic transducer of the array of ultrasonic transducers selected as exhibiting alignment with the target exhibits the smallest signal amplitude of the signal amplitudes of each instance of the ultrasonic scanning operation. In some embodiments, as shown at procedure 1240, the ultrasonic transducer of the array of ultrasonic transducers selected as exhibiting alignment with the target exhibits the largest time of flight differential between a first reflected ultrasonic signal of the at least one reflected ultrasonic signal and a second reflected ultrasonic signal of the at least one reflected ultrasonic signal. In some embodiments, as shown at procedure 1242, the ultrasonic transducer of the array of ultrasonic transducers selected as exhibiting alignment with the target exhibits the largest signal amplitude differential for each sub-array. In some embodiments, as shown at procedure 1244, the ultrasonic transducer of the array of ultrasonic transducers selected as exhibiting alignment with the target exhibits the largest time of flight differential between reflected ultrasonic signals received during two instances of the ultrasonic scanning operation for each sub-array. In some embodiments, as shown at procedure 1246, the ultrasonic transducer of the array of ultrasonic transducers selected as exhibiting alignment with the target exhibits largest velocity amplitude of a Doppler signal. In some embodiments, as shown at procedure 1248, the ultrasonic transducer of the array of ultrasonic transducers selected as exhibiting alignment with the target exhibits largest width of the velocity profile identified from a Doppler signal.

### CONCLUSION

The examples set forth herein were presented in order to best explain, to describe particular applications, and to thereby enable those skilled in the art to make and use embodiments of the described examples. However, those skilled in the art will recognize that the foregoing description and examples have been presented for the purposes of illustration and example only. Many aspects of the different example embodiments that are described above can be combined into new embodiments. The description as set forth is not intended to be exhaustive or to limit the embodiments to the precise form disclosed. Rather, the specific features and acts described above are disclosed as example forms of implementing the claims.

Reference throughout this document to “one embodiment,” “certain embodiments,” “an embodiment,” “various embodiments,” “some embodiments,” or similar term means that a particular feature, structure, or characteristic described in connection with the embodiment is included in at least one embodiment. Thus, the appearances of such phrases in various places throughout this specification are not necessarily all referring to the same embodiment. Furthermore, the particular features, structures, or characteristics of any embodiment may be combined in any suitable manner with one or more other features, structures, or characteristics of one or more other embodiments without limitation.

What is claimed is:

1. A wearable system for cardiovascular monitoring, the system comprising:
an ultrasonic sensor module comprising at least one array of ultrasonic transducers and at least one pre-amplification device coupled to the at least one array of ultrasonic transducers, the at least one array of ultrasonic transducers comprising a plurality of ultrasonic transducers, the ultrasonic sensor module for placement on a human body proximate a blood vessel for performing cardiovascular monitoring;
a data storage unit;
a processing unit comprising:
hardware componentry for controlling transmission of ultrasonic signals at the at least one array of ultrasonic transducers and for controlling receipt of reflected ultrasonic signals at the at least one array of ultrasonic transducers;
a digital processing module for performing on-board signal processing of the reflected ultrasonic signals received at the at least one array of ultrasonic transducers; and
a power control system comprising an energy storage device for providing power to the hardware componentry, the digital processing module, and the ultrasonic sensor module; and
a connection cable assembly for communicatively coupling the ultrasonic sensor module to the processing unit, the connection cable assembly for enabling signal communication and power transmission between the ultrasonic sensor module and the processing unit.

2. The system of claim 1, wherein the ultrasonic sensor module comprises a first array of ultrasonic transducers and a second array of ultrasonic transducers.

3. The system of claim 2, wherein the first array of ultrasonic transducers and the second array of ultrasonic transducers are one-dimensional arrays of ultrasonic transducers.

4. The system of claim 1, wherein the ultrasonic sensor module further comprises a motion sensor for performing motion sensing, wherein the hardware componentry is also for controlling operation of the motion sensor.

5. The system of claim 4, wherein the digital processing module is also for correlating motion sensing data of the motion sensor with the reflected ultrasonic signals for use in the signal processing of the reflected ultrasonic signals.

6. The system of claim 4, wherein the hardware componentry is for controlling transmission of the ultrasonic signals according to motion sensing data of the motion sensor.

7. The system of claim 1, wherein the ultrasonic sensor module further comprises a force sensor, wherein the hardware componentry is also for controlling operation of the force sensor, wherein the force sensor is for sensing an applied pressure between the ultrasonic sensor module and the human body for ensuring appropriate contact pressure between the ultrasonic sensor module and the human body.

8. The system of claim 7, wherein the digital processing module also utilizes the applied pressure in performing the cardiovascular monitoring.

9. The system of claim 1, wherein the connection cable assembly comprises a plurality of signal lines corresponding to each transmission signal and each received signal for each receive channel of the at least one array of ultrasonic transducers.

10. The system of claim 1, wherein the digital processing module further comprises a wireless communication module for communicating output of the on-board signal processing to a remote computer system.

11. The system of claim 1, wherein the hardware componentry, the digital processing module, and the power control system of the processing unit are comprised within a system on a chip (SoC) comprising a processor and a field programmable gate array (FPGA) for performing the on-board signal processing.

12. The system of claim 1, wherein the ultrasonic sensor module comprises a flex-board sub-unit comprising the at least one array of ultrasonic transducers and a rigid board comprising the at least one pre-amplification device.

13. The system of claim 1, wherein the ultrasonic sensor module further comprises a light emitting diode for providing visual feedback for aligning the at least one array of ultrasonic transducers with a blood vessel.

14. The system of claim 1, wherein the digital processing module is also for aligning the at least one array of ultrasonic transducers by selecting an ultrasonic transducer of the at least one array of ultrasonic transducers as an aligned ultrasonic transducer.

15. The system of claim 1, wherein the cardiovascular monitoring comprises at least one of pulse wave velocity, pulse transit time, arterial diameter, arterial wall motion, arterial wall stiffness, heart rate, and blood pressure.

16. The system of claim 1, further comprising a second ultrasonic sensor module for placement on the human body at a different location than the ultrasonic sensor module and proximate a blood vessel for performing cardiovascular monitoring, wherein the processing unit is configured to trigger the hardware componentry of the ultrasonic sensor module and the second ultrasonic sensor module for synchronization of the transmission of the ultrasonic signals and the receipt of the reflected ultrasonic signals between the ultrasonic sensor module and the second ultrasonic sensor module.

17. The system of claim 1, further comprising a second processing unit coupled to a second ultrasonic sensor module for placement on the human body at a different location than the ultrasonic sensor module and proximate a blood vessel for performing cardiovascular monitoring, wherein the processing unit and the second processing unit are configured to coordinate synchronous clocking via a synchronization signal.

18. The system of claim 1, the processing unit further comprising an activation timer configured for autonomous activation of the ultrasonic sensor module and the processing unit enabling the transmission of the ultrasonic signals and the receipt of the reflected ultrasonic signals.

19. A wearable system for cardiovascular monitoring, the system comprising:
an ultrasonic sensor module comprising a first array of ultrasonic transducers and a second array of ultrasonic transducers, a first pre-amplification device coupled to the first array of ultrasonic transducers, a second pre-amplification device coupled to the second array of ultrasonic transducers, the first array of ultrasonic transducers and the second array of ultrasonic transducers comprising a plurality of ultrasonic transducers, the ultrasonic sensor module for placement on a human body proximate a blood vessel for performing cardiovascular monitoring;
a data storage unit;
a processing unit comprising:
hardware componentry for controlling transmission of ultrasonic signals at the first array of ultrasonic transducers and the second array of ultrasonic transducers and for controlling receipt of reflected ultrasonic signals at the first array of ultrasonic transducers and the second array of ultrasonic transducers;
a digital processing module for performing on-board signal processing of the reflected ultrasonic signals received at the first array of ultrasonic transducers and the second array of ultrasonic transducers and for aligning the first array of ultrasonic transducers and the second array of ultrasonic transducers by selecting an ultrasonic transducer of the first array of ultrasonic transducers as an aligned ultrasonic transducer of the first array of ultrasonic transducers and selecting an ultrasonic transducer of the second array of ultrasonic transducers as an aligned ultrasonic transducer of the second array of ultrasonic transducers; and
a power control system comprising an energy storage device for providing power to the hardware componentry, the digital processing module, and the ultrasonic sensor module; and
a connection cable assembly for communicatively coupling the ultrasonic sensor module to the processing unit, the connection cable assembly for enabling signal communication and power transmission between the ultrasonic sensor module and the processing unit.

20. A wearable system for cardiovascular monitoring, the system comprising:
an ultrasonic sensor module comprising at least one array of ultrasonic transducers, at least one pre-amplification device coupled to the at least one array of ultrasonic transducers, and a motion sensor for performing motion sensing, the at least one array of ultrasonic transducers comprising a plurality of ultrasonic transducers, the ultrasonic sensor module for placement on a human body proximate a blood vessel for performing cardiovascular monitoring;
a data storage unit;
a processing unit comprising:
hardware componentry for controlling transmission of ultrasonic signals at the at least one array of ultrasonic transducers, for controlling receipt of reflected ultrasonic signals at the at least one array of ultrasonic transducers according to motion sensing data of the motion sensor, and for controlling operation of the motion sensor;
a digital processing module for performing on-board signal processing of the reflected ultrasonic signals received at the at least one array of ultrasonic transducers and for correlating the motion sensing data of the motion sensor with the reflected ultrasonic signals for use in the signal processing of the reflected ultrasonic signals; and
a power control system comprising an energy storage device for providing power to the hardware componentry, the digital processing module, and the ultrasonic sensor module; and
a connection cable assembly for communicatively coupling the ultrasonic sensor module to the processing unit, the connection cable assembly for enabling signal communication and power transmission between the ultrasonic sensor module and the processing unit.
